SEQUENCE LISTING (6 sequences)

SEQ ID NO: 1 - protein, 10 aa, Homo sapiens
QHWSYKLRPA

SEQ ID NO: 2 - protein, 9 aa, Homo sapiens
QHWSYKLRP

SEQ ID NO: 3 - protein, 10 aa, Homo sapiens
QHWSYCLRPA

SEQ ID NO: 4 - protein, 9 aa, Homo sapiens
QHWSYCLRP

SEQ ID NO: 5 - protein, 10 aa, Homo sapiens
QHWSYELRPA

SEQ ID NO: 6 - protein, 9 aa, Homo sapiens
QHWSYELRP

(19) United States
(12) Patent Application Publication
(10) Pub. No.: US 2024/0082409 A1
(43) Pub. Date: Mar. 14, 2024

(54) Modified peptide ligands for stable delivery of highly potent payloads to tumors: Method of making and using same

(71) Applicant: NAJIB LAMHARZI, Bothell, WA (US)

(72) Inventor: NAJIB LAMHARZI, Bothell, WA (US)

(21) Appl. No.: 17/525,040

(22) Filed: Oct. 17, 2023

Related U.S. Application Data
(60) Provisional application No. 63/114,208, filed on Nov. 16, 2020.

(51) Int. Cl. A61K 47/64; A61K 47/60; A61K 47/65; A61P 35/00; C07K 7/06

(52) U.S. Cl. CPC A61K 47/64; A61K 47/60; A61K 47/65; A61P 35/00; C07K 7/06

(57) ABSTRACT

The conjugation of luteinizing hormone-releasing hormone analogs (LHRHa) to highly potent payloads via spacers (linear or branched) and cleavable peptidic linkers generate peptide drug conjugates (PDCs) with stable triazole, thioether, oxime or amide bonds. The PDCs target tumor cells that express LHRH receptor (LHRH-R) and release their payloads inside the lysosomes. The PDCs may be used in a method for killing or inhibiting the growth of a tumor cell, especially in late state, highly invasive and aggressive stage IV tumors and in reoccurring tumors.

### OTHER REFERENCES

Harrison, G., et al., “Gonadotropin-releasing hormone and its receptor in normal and malignant cells” Endocr. Relat. Cancer, 2004, pp. 725-48 (11).
Lamharzi, N., et al., “Expression of mRNA for luteinizing hormone-releasing hormone receptors and epidermal growth factor receptors in human cancer cell lines” Int. J. Oncol. 1998, pp. 671-675, 12.
Halmos, G., et al., “High incidence of receptors for luteinizing hormone-releasing hormone (LHRH) and LHRH receptor gene expression in human prostate cancers”. J Urol., 2000, pp. 623-9, 163.
Tieva, A., et al., “Gonadotropin-releasing hormone receptor expression in the human prostate” Prostate, 2001, pp. 276-84, 47.
Fekete, M., et al., “Characteristics and distribution of receptors for [D-TRP⁶]-luteinizing hormone-releasing hormone, somatostatin, epidermal growth factor, and sex steroids in 500 biopsy samples of human breast cancer,” Journal of clinical laboratory analysis, 1989, pp. 137-147, 3.
Buchholz, S., et al., “Triple-negative breast cancers express receptors for luteinizing hormone-releasing hormone (LHRH) and respond to LHRH antagonist cetrorelix with growth inhibition” Int J Oncol. pp. 789-796, 35.
Kwok, C., et al., “Receptors for luteinizing hormone-releasing hormone (GnRH) as therapeutic targets in triple negative breast cancers (TNBC)” 2015, pp. 365-73, 10.
Fekete M., et al., “Membrane receptors for peptides in experimental and human pancreatic cancers” Pancreas, 1989, pp. 521-8, 4.
Friess, H., et al., “LH-RH receptors in the human pancreas. Basis for antihormonal treatment in ductal carcinoma of the pancreas” Int. J Pancreatol., 19991, pp. 151-9, 10.
Sion-Vardi N., et al., “Gonadotropin-releasing hormone specific binding sites in normal and malignant renal tissue”. J Urol., 1992, pp. 1568-70, 148.
Engel, J., et al., “AEZS-108: A Targeted Cytotoxic Analog of LHRH for the Treatment of Cancers Positive for LHRH Receptors”. Expert. Opin. Investig. Drugs, 2012, pp. 891-9, 21.
Engel, J., et al., “Targeted chemotherapy of endometrial, ovarian and breast cancers with cytotoxic analogs of luteinizing hormone-releasing hormone (LHRH). Arch Gynecol. Obstet. 2012, pp. 437-42, 286.
Nagy A., et al., “Targeting of cytotoxic luteinizing hormone-releasing hormone analogs to breast, ovarian, endometrial, and prostate cancers. Biol. Reprod. 2005, pp. 851-9, 73.
Halmos G., et al., “Cytotoxic analogs of luteinizing hormone-releasing hormone bind with high affinity to human breast cancers” Cancer Lett. 1999, pp. 129-36, 136.
Kahan Z., et al., “Complete regression of MX-1 human breast carcinoma xenografts after targeted chemotherapy with a cytotoxic analog of luteinizing hormone-releasing hormone, AN-207” Cancer 1999, pp. 2608-15, 85.
Bajo, A., et al., “Targeted doxorubicin-containing luteinizing hormone-releasing hormone analogue AN-152 inhibits the growth of doxorubicin-resistant MX-1 human breast cancers” Clin. Cancer Res., 2003, pp. 3742-8, 9.
Föst, C., et al., “Targeted Chemotherapy for Triple-Negative Breast Cancers via LHRH Receptor”. Oncol. Rep. 2011, pp. 1481-7, 25.
Popovics P., et al., “Targeted cytotoxic analog of luteinizing hormone-releasing hormone (LHRH), AEZS-108 (AN-152), inhibits the growth of DU-145 human castration-resistant prostate cancer in vivo and in vitro through elevating p21 and ROS levels” Oncotarget. 2014, pp. 4567-4578, 5.
Szepeshazi K., et al., “Receptor-targeted therapy of human experimental urinary bladder cancers with cytotoxic LH-RH analog AN-152 [AEZS-108]”. Oncotarget, 2012, pp. 686-99, 3.
Gründker C., et al., “Effective targeted chemotherapy using AEZS-108 (AN-152) for LHRH receptor-positive pancreatic cancers” Oncol. Rep., 2011, pp. 629-35, 26.
Nagy, A., et al., “Stability of cytotoxic luteinizing hormone-releasing hormone conjugate (AN-152) containing doxorubicin 14-O-hemiglutarate in mouse and human serum in vitro: implications for the design of preclinical studies” Proc Natl Acad Sci USA., 2000, pp. 829-34, 97.
Kovacs M., et al., “Recovery of pituitary function after treatment with a targeted cytotoxic analog of luteinizing hormone releasing hormone” Proc Natl Acad Sci USA., 1997, pp. 1420-5, 94.
Nagy, A., et al., “Cytotoxic analogs of luteinizing hormone-releasing hormone containing doxorubicin or 2-pyrrolinodoxorubicin, a derivative 500-1000 times more potent” Proc Natl Acad Sci USA., 1996, pp. 7269-73, 93.
Firestone, R., et al., “Lysosomal enzyme-cleavable antitumor drug conjugates. Patents EP0624377A2 and U.S. Pat. No. 6,214,345B1, 1994, 2001.
Francisco, J., et al., “cAC10-vcMMAE, an anti-CD30-monomethyl auristatin E conjugate with potent and selective activity”. Blood, 2003, pp. 1458-1465, 102.
Danila, D., et al., “Selective induction of apoptosis by the cytotoxic analog AN-207 in cells expressing recombinant receptor for luteinizing hormone-releasing hormone” Proc. Natl. Acad. Sci. USA., 1999, pp. 669-73, 96.
Okada, Y., et al., “Evidence that gonadotropin-releasing hormone (GnRH) II stimulates luteinizing hormone and follicle-stimulating hormone secretion from monkey pituitary cultures by activating the GnRH I receptor” Biol. Reprod. 2003, pp. 1356-1361, 69.
Bajusz S., et al., “New antagonists of LHRH. II. Inhibition and potentiation of LHRH by closely related analogues” Int. J. Pept. Protein Res. 1988, pp 425-435, 32.
Proudman, J. A., et al. “Comparison of the ability of the three endogenous GnRHs to stimulate release of follicle-stimulating hormone and luteinizing hormone in chickens” Domest. Anim. Endocrinol. 2006, pp. 141-153, 31.
Sower, S. A., et al., “Primary structure and biological activity of a third gonadotropin-releasing hormone from lamprey brain” Endocrinology, 1993, pp. 1125-1131, 132.
Kovacs, M., et al., “Lamprey gonadotropin hormone-releasing hormone-III has no selective follicle-stimulating hormone releasing effect in rats” J. Neuroendocrinol., 2002, pp. 647-655, 14.
Szabó, I., et al., “Development of an oxime bond containing daunorubicin gonadotropin-releasing hormone-III conjugate as a potential anticancer drug” Bioconju. Chem, 2009, pp. 656-665, 20.
Leurs, U., et al., “In vitro stability and cytostatic effect of multifunctional anticancer drug-bioconjugates containing GnRH III as a targeting moiety” Biopolymers, 2012, pp. 1-10, 98.
Jewett, J. C.; et al., Rapid Cu-Free Click Chemistry with Readily Synthesized Biarylazacyclooctynones. J. Am. Chem. Soc. 2010, pp. 3688-3690, 132.
Chang, P. V., et al., Copper-free click chemistry in living animals. Proc. Natl. Acad. Sci. 2010, pp. 1821-1826, 107.
Hashida, S., et al. More useful maleimide compounds for the conjugation of Fab to horseradish peroxidase through thiol groups in the hinge. J Appl Biochem., 1984, pp. 56-63, 6.
Yoshitake, S., et al. Mild and efficient conjugation of rabbit Fab and horseradish peroxidase using a maleimide compound and its use for enzyme immunoassay. J Biochem., 1982, pp. 1413-24, 92 Grabarek, Z. and Gergely, J. Zero-length crosslinking procedure with the use of active esters. Anal Biochem., 1990, pp. 131-5, 185.
Staros, J. V., et al. Enhancement by N-hydroxysulfosuccinimide of water-soluble carbodiimide-mediated coupling reactions. Anal Biochem., 1986, pp. 220-2, 156.
Timkovich, R. Detection of the stable addition of carbodiimide to proteins. Anal Biochem., 1977, pp. 135-43, 79.

### DESCRIPTION

### Field of the Disclosure

The present invention relates to treating and preventing cancers, more particularly to LHRHa-based drug conjugates for treating cancers through the stable delivery of specific potent payloads to a tumor cell expressing the target receptor.

### Discussion of Related Art

LHRH and LHRH Receptor:

Various studies have found that in comparison to normal cells, receptors for certain peptides/hormones are found in higher concentration in tumor cells than in normal cells. For example, the receptor for luteinizing hormone releasing hormone (LHRH-R) is over-expressed in numerous types of cancer cells including breast, ovarian, endometrial, prostate, cervical, pancreatic and kidney cancers as well as glioblastomas, making the LHRH peptide a good candidate for drug targeting. Even though several LHRH-based drug conjugates have been studied in animal models, only AEZS-108 reached but failed Phase 3 clinical trial due to the low potency of the doxorubicin, the low stability of the ligand and the low stability of the ester bond linking the ligand to doxorubicin (2 hours halve-life). See Lamharzi, N. et al. “Expression of mRNA for luteinizing hormone-releasing hormone receptors and epidermal growth factor receptors in human cancer cell lines”. Int. J. Oncol., 1998, pp. 671-675, 12; Harrison, G. et al., “Gonadotropin-releasing hormone and its receptor in normal and malignant cells”. Endocr. Relat. Cancer 11, 2004, pp. 725-48; Halmos, G. et al. “Cytotoxic analogs of luteinizing hormone-releasing hormone bind with high affinity to human breast cancers”. Cancer Lett., 1999, pp. 129-36, 136; Buchholz, S. et al. “Triple-negative breast cancers express receptors for luteinizing hormone-releasing hormone (LHRH) and respond to LHRH antagonist cetrorelix with growth inhibition”. Int J Oncol., 2009, 35, pp. 789-796, 35; Kwok C. et al. “Receptors for luteinizing hormone-releasing hormone (GnRH) as therapeutic targets in triple negative breast cancers (TNBC)”. Target Oncol., 2015, pp. 365-73, 10; Popovics, P. et al. “Targeted cytotoxic analog of luteinizing hormone-releasing hormone (LHRH), AEZS-108 (AN-152), inhibits the growth of DU-145 human castration-resistant prostate cancer in vivo and in vitro through elevating p21 and ROS levels”. Oncotarget, 2014, pp. 4567-4578, 5; Schally, A. V. et al., “Cancer chemotherapy based on targeting of cytotoxic peptide conjugates to their receptors on tumors” Eur. J. Endocrinol., 1999, pp. 1-14, 141; Nagy, A. et al., “Cytotoxic analogs of luteinizing hormone-releasing hormone (LHRH): a new approach to targeted chemotherapy,” Drug Future, 2002, pp. 359-370, 27; Nagy, A. et al., “Targeting cytotoxic conjugates of somatostatin, luteinizing hormone-releasing hormone and bombesin to cancers expressing their receptors: a “smarter”chemotheraphy,” Curr. Pharm. Des., 2005, pp. 1167-1180, 11; Schally, A. V. et al., “New approaches to treatment of various cancers based on cytotoxic analogs of LHRH, somatostatin and bombesin,” Life Sci., 2003, pp. 2305-2320, 72; Emons, G. et al., “The use of luteinizing hormone releasing hormone agonists and antagonists in gynecological cancers,” Hum Reprod., 1994, pp. 1364-1379, 9; Engel, J. et al., “Targeted chemotherapy of endometrial, ovarian and breast cancers with cytotoxic analogs of luteinizing hormone-releasing hormone (LHRH)”. Arch. Gynecol. Obstet., 2012, pp. 437-42, 286; Engel J. et al., “AEZS-108: A targeted cytotoxic analog of LHRH for the treatment of cancers positive for LHRH receptors” Expert Opin. Investig. Drugs, 2012, pp. 891-9, 21; Nagy A. et al. “Targeting of cytotoxic luteinizing hormone-releasing hormone analogs to breast, Ovarian, Endometrial, and Prostate Cancers” Biol. Reprod., 2005, pp. 851-9, 73; Gründker C. et al., “Effective Targeted Chemotherapy Using AEZS-108 (AN-152) for LHRH Receptor-Positive Pancreatic Cancers”. Oncol. Rep., 2011, pp. 629-35, 26; Duwe F. et al., “Targeted chemotherapy for triple-negative breast cancers via LHRH receptor”. Oncol. Rep., 2011, pp. 1481-7, 25; Szepeshazi K. et al. “Receptor-targeted therapy of human experimental urinary bladder cancers with cytotoxic LH-RH analog AN-152 [AEZS-108]”. Oncotarget, 2012, pp. 686-99, 3; Nagy A. et al., “Stability of cytotoxic luteinizing hormone-releasing hormone conjugate (AN-152) containing doxorubicin 14-O-hemiglutarate in mouse and human serum in vitro: implications for the design of preclinical studies. Proc Natl Acad Sci USA., 2000, 829-34, 97.

In addition to LHRH-I, it has been reported that 2 other forms of LHRH (II and III) have been identified and shown to bind to LHRH-I receptor (Okada, Y., et al., “Evidence that gonadotropin-releasing hormone (GnRH) II stimulates luteinizing hormone and follicle-stimulating hormone secretion from monkey pituitary cultures by activating the GnRH I receptor” Biol. Reprod. 2003, pp. 1356-1361, 69; Bajusz S., et al., “New antagonists of LHRH. II. Inhibition and potentiation of LHRH by closely related analogues” Int. J. Pept. Protein Res. 1988, pp 425-435, 32; Proudman, J. A., et al. “Comparison of the ability of the three endogenous GnRHs to stimulate release of follicle-stimulating hormone and luteinizing hormone in chickens” Domest. Anim. Endocrinol. 2006, pp. 141-153, 31; Sower, S. A., et al., “Primary structure and biological activity of a third gonadotropin-releasing hormone from lamprey brain” Endocrinology, 1993, pp. 1125-1131, 132; Kovacs, M., et al., “Lamprey gonadotropin hormone-releasing hormone-III has no selective follicle-stimulating hormone releasing effect in rats” J. Neuroendocrinol., 2002, pp. 647-655, 14; Szabó, I., et al., “Development of an oxime bond containing daunorubicin gonadotropin-releasing hormone-III conjugate as a potential anticancer drug” Bioconju. Chem, 2009, pp. 656-665, 20; Leurs, U., et al., “In vitro stability and cytostatic effect of multifunctional anticancer drug-bioconjugates containing GnRH III as a targeting moiety” Biopolymers, 2012, pp. 1-10, 98.

Because previous LHRH-based drug conjugates have several disadvantages including the low stability of the linker and low potency of the payload we have manufactured stable and highly potent LHRH-based conjugates for treating cancers. Such LHRH-based conjugates would decrease adverse side effects associated with existing drugs, and limit nonspecific activity. Here, we designed stable LHRH ligands allowing conjugation through their amino acid in position 4, 6 or 8 to highly potent cytotoxic agents (picomolar to nanomolar in-vitro potency) through specific spacers and linkers. The conjugation generate either a triazole bond between D-Lys[N₃]⁴, D-Lys[N₃]⁶ or D-Lys[N₃]⁸ of LHRHa and DBCO of the spacer; thioether bond between the SH group of [D-Cys⁴]LHRHa, [D-Cys⁶]LHRHa or [D-Cys⁸]LHRHa and Maleimide group of the spacer; amide bond between NH₂ side chain group of [D-Lys⁴]LHRHa, [D-Lys⁶]LHRHa or [D-Lys⁸]LHRHa and NHS ester group of the spacer; amide bond between the activated COOH side chain group of [D-Glu⁴]LHRHa, [D-Asp⁴]LHRHa, [D-Glu⁶]LHRHa, [D-Asp⁶]LHRHa, [D-Glu⁸]LHRHa or [D-Asp⁸]LHRHa and NH₂ group of the spacer-linker-payload. The linkers used between the spacers and the payloads are either Val-Cit-PABC, Val-Ala, Gly-Gly-Phe-Gly or other highly stable peptide linkers cleaveable by cathepsins inside the lysosomes of cancer cells. For example we generated several monomeric LHRH-based drug conjugates carrying monomethyl auristatin E (MMAE) which inhibit tubulin polymerization and it is highly potent than doxorubicin. Our LHRH-based drug conjugates including OTS-P22, OTS-P23, OTS-P23b, OTS-P26, OTS-P28 and OTS-P31 showed higher potency at low nanomolar concentrations in-vitro. In-vivo, OTS-P22 at a single injection of 1.435 mg/kg caused a 100% tumor growth inhibition of HCC1806 triple-negative breast cancer (TNBC) xenografted in SCID mice while unconjugated MMAE did not show any significant tumor growth inhibition at the end of the 21 days study. As compared to the first generation LHRH drug conjugate AEZS-108 which failed clinical trials due to low stability and low potency, OTS-P22 is at least 16 times more potent in term of in-vivo tumor growth inhibition of HCC1806 xenografts. No signifcant effect of OTS-P22 on animal weight, hematology and normal tissue histology was observed. We have also generated additional drug conjugates including OTS-P28 which is highly stable in human plasma and has anti-proliferative activity in-vitro at low nanomolar concentrations on HCC1806 TNBC cell line. Furthermore, we manufactured another LHRH-based drug conjugate OTS-P31 by conjugation through the NH₂ side chain amino acid of D-Lysine in position 6 of LHRH analog to NHS ester-Glut-Val-Cit-PABC-MMAE (Glut is glutaric acid). Copper-free click chemistry, click chemistry or NHS ester conjugation to amine group used to manufacture our LHRHa conjugates are based on established protocols. See Jewett J C., et al., “Rapid Cu-Free Click Chemistry with Readily Synthesized Biarylazacyclooctynones” J. Am. Chem. Soc. 2010, pp. 3688-3690, 132; Chang P V., et al., “Copper-free click chemistry in living animals” Proc. Natl. Acad. Sci. 2010, pp. 1821-1826, 107; Hashida S., et al., “More useful maleimide compounds for the conjugation of Fab to horseradish peroxidase through thiol groups in the hinge” J Appl. Biochem., 1984, pp. 56-63, 6; Yoshitake S., et al., “Mild and efficient conjugation of rabbit Fab and horseradish peroxidase using a maleimide compound and its use for enzyme immunoassay” J Biochem., 1982, pp. 1413-24, 92; Grabarek Z. and Gergely, J., “Zero-length crosslinking procedure with the use of active esters” Anal Biochem., 1990, pp. 131-5, 185; Staros J V., et al., “Enhancement by N-hydroxysulfosuccinimide of water-soluble carbodiimide-mediated coupling reactions” Anal Biochem., 1986, pp. 220-2, 156; Timkovich R “Detection of the stable addition of carbodiimide to proteins” Anal Biochem., 1977, pp. 135-43, 79.

Here are the examples of our monomeric LHRH-I ligands used for conjugation to generate drug conjugates OTS-P22, OTS-P23, OTS-P23b, OTS-P26, OTS-P28 and OTS-P31:

Conjugation of LHRH-I ligand with the following structure Glp-His-DPal-Ser-Tyr-DLys[N₃]-Leu-Arg-Pro-DAla-NH₂ to DBCO-dPEG₃-Val-Cit-PABC-MMAE by means of copper-free click chemistry generates OTS-P22 with the structure Glp-His-DPal-Ser-Tyr-DLys[dPEG₃-Val-Cit-PABC-MMAE]-Leu-Arg-Pro-DAla-NH₂.

Conjugation of LHRH-I ligand with the following structure Glp-His-DPal-Ser-Tyr-DLys[N₃]-Leu-Arg-Pro-DAla-NH₂ to DBCO-dPEGg-Val-Cit-PABC-MMAE by means of copper-free click chemistry generates OTS-P23 with the structure Glp-His-DPal-Ser-Tyr-DLys[dPEG₈-Val-Cit-PABC-MMAE]-Leu-Arg-Pro-DAla-NH₂.

Conjugation of LHRH-I ligand Glp-His-Trp-DSer-Tyr-DLys[N₃]-Leu-Arg-Pro-NHEt to DBCO-dPEG₅-Val-Cit-PABC-MMAE by means of copper-free click chemistry generates OTS-P23b with the structure Glp-His-Trp-DSer-Tyr-DLys[-dPEG₅-Val-Cit-PABC-MMAE]-Leu-Arg-Pro-NHEt.

Conjugation of LHRH-I ligand Glp-His-DPal-Ser-Tyr-DCys-Leu-Arg-Pro-DAla-NH₂ to MC-Val-Cit-PABC-MMAE by means of click chemistry generates OTS-P26 with the structure Glp-His-DPal-Ser-Tyr-DCys[Val-Cit-PABC-MMAE]-Leu-Arg-Pro-DAla-NH₂.

Conjugation of LHRH-I ligand Glp-His-DPal-Ser-Tyr-DCys-Leu-Arg-Pro-DAla-NH₂ to Mal-dPEG₈-Val-Cit-PABC-MMAE by means of click chemistry generates OTS-P28 with the structure Glp-His-DPal-Ser-Tyr-DCys[dPEG₈-Val-Cit-PABC-MMAE]-Leu-Arg-Pro-DAla-NH₂.

Conjugation of LHRH-I ligand Glp-DHis-Trp-DSer-Tyr-DLys-Leu-Arg-Pro-DAa-NH₂ to NHS ester-Glut-Val-Cit-PABC-MMAE through amino side chain of DLysine and NHS ester group to generate OTS-P31 with the structure Glp-DHis-Trp-DSer-Tyr-DLys[Glut-Val-Cit-PABC-MMAE]-Leu-Arg-Pro-DAla-NH₂.

Additional examples using ligands, linkers and payloads described in claim 1, 2, 3, 4, 5 and 6 are expected to generate highly stable and potent LHRH-based drug conjugates against solid and hematological cancers.

### SUMMARY OF THE DISCLOSURE

The present invention describes the amino acid composition and conjugation of LHRH analogs through amino acids in position 4, 6 or 8 of [DLys^{4(N3)}]LHRHa, [D-Lys⁴]LHRHa, [DLys⁴-Aoa], [D-Cys⁴]LHRHa, [D-Glu⁴]LHRHa, [D-Asp⁴]LHRHa, [DLys^{6(N3)}]LHRHa, [D-Lys⁶]LHRHa, [DLys⁶-Aoa], [D-Cys⁶]LHRHa, [D-Glu⁶]LHRHa, [D-Asp⁶]LHRHa, [DLys^{8(N3)}]LHRHa, [D-Lys-]LHRHa, [DLys⁶-Aoa], [D-Cys⁸]LHRHa, [D-Glu⁸]LHRHa, [D-Asp⁸]LHRHa. Conjugation of LHRH analogs with the terminal end of Spacer-Linker-Payload gives rise to new and stable peptide drug conjugates that effectively targets tumor cells expressing the LHRH receptor. The present embodiment overcomes the existing shortcomings in this area by accomplishing critical objectives particularly the high stability, in-vivo potency and safety of the conjugates. The LHRH-based drug conjugates are intended to be used in the treatment of reproductive and non-reproductive cancers expressing the LHRH receptor. The use of the LHRH-based drug conjugates will increase the concentration of the highly potent payload in tumor cells and attenuate unnecessary exposure to normal cells. The LHRH-based drug conjugates have applications to breast cancer including triple-negative breast cancer, ovarian cancer, endometrial cancer, cervical and prostate cancer as well as other metastatic cancers expressing the LHRH receptor such as kidney and pancreatic cancers, especially in late stage, highly invasive and aggressive stage IV tumors, and in reoccurring tumors as these are the most difficult to treat. These peptide drug conjugates are more suitable for treating solid tumors due to their small size (˜3 KDa) and thus could readily penetrate the tumor mass as compared to antibody drug conjugates which are larger (˜150 KDa). Other features and advantages of the present invention will become apparent from the following more detailed description, taken in conjunction with the accompanying drawings, which illustrate, by way of example, the principles of the invention.

The binding moiety targets cells for destruction by the payload, including undesirable or aberrant proliferating cells or hyperproliferating cells, such as non-metastatic and metastatic neoplasias, cancers, tumors and malignancies. A number of non-metastatic and metastatic neoplastic, cancer, tumor and malignant cells overexpress receptors for LHRH that can be used as binding moiety of the LHRH-based conjugates. The binding moiety could be LHRH-I, LHRH-II or LHRH-III in monomeric or multimeric form.

Drug conjugates include or consist of purified forms or of a mixture, such as a mixture of drug conjugate and a pharmaceutically acceptable carrier or excipient appropriate for administration to or in vivo contact with a subject, or a mixture of drug conjugates and or immune stimulating agents.

Drug conjugates include or consist of a unit dosage form. In one embodiment, a drug conjugate is a unit dosage in an amount effective to treat a subject having undesirable cell proliferation or a hyperproliferative disorder. In another embodiment, a drug conjugate is a unit dosage in an amount effective to treat a subject having a neoplasia, tumor or cancer.

Drug conjugates can be included within kits, optionally with instructions for practicing a method. In one embodiment, a kit includes a drug conjugate and instructions for treating a subject having a hyperproliferative disorder, treating a subject having a neoplasia, tumor or cancer.

Drug conjugates are useful for, among other things, reducing or inhibiting proliferation of a cell, reducing or inhibiting cell proliferation, reducing or inhibiting proliferation of a hyperproliferating cell, reducing or inhibiting proliferation of a neoplastic, tumor, cancer or malignant cell and treating undesirable or aberrant cell proliferation, such as hyperproliferating cells or hyperproliferative disorders. Non-limiting examples of hyperproliferative disorders include benign hyperplasia, non-metastatic and metastatic neoplasias, cancers tumors and malignancies (e.g., a solid or liquid tumor, myeloma, lymphoma, leukemia, carcinoma, sarcoma, melanoma, neural, reticuloendothelial and haematopoietic).

In accordance with the invention, there are further provided methods of reducing or inhibiting proliferation of a cell; methods of reducing or inhibiting proliferation of a hyperproliferating cell; and methods of reducing or inhibiting proliferation of a neoplastic, tumor, cancer or malignant cell. In various embodiments, a method includes contacting a cell with a drug conjugate in an amount sufficient to reduce or inhibit proliferation of the cell; contacting a cell with a drug conjugate in an amount sufficient to reduce or inhibit cell proliferation; contacting a cell with a drug conjugate in an amount sufficient to reduce or inhibit proliferation of the hyperproliferating cell; and contacting a cell with a drug conjugate in an amount sufficient to reduce or inhibit proliferation of the neoplastic, tumor, cancer or malignant cell.

In accordance with the invention, there are moreover provided methods of selectively reducing or inhibiting proliferation of a cell that expresses a receptor; selectively reducing or inhibiting proliferation of a hyperproliferating cell that expresses a receptor; and selectively reducing or inhibiting proliferation of a neoplastic, tumor, cancer or malignant cell that expresses a receptor. In various embodiments, a method includes contacting a cell with the drug conjugate in an amount sufficient to reduce or inhibit proliferation of the cell, wherein the binding moiety of said ligand binds to the receptor expressed by the cell; contacting a cell with the drug conjugate in an amount sufficient to reduce or inhibit proliferation of the hyperproliferating cell, wherein the binding moiety of said ligand binds to the receptor expressed by the hyperproliferating cell; and contacting a cell with the drug conjugate in an amount sufficient to reduce or inhibit proliferation of the neoplastic, tumor, cancer or malignant cell, wherein the binding moiety of said drug conjugate binds to the receptor expressed by the cell.

Cells targeted in accordance with the invention methods include cells that express a receptor, for example, Luteinizing hormone releasing hormone receptor (LHRH-R). Cells targeted in accordance with the invention methods also include cells that express a receptor that binds to LHRH-I, LHRH-II or LHRH-III.

Methods performed include, among others, contacting a subject in need of inhibiting, reducing or preventing proliferation, survival, differentiation, death, or activity of a cells, such as a hyperproliferative cell or an undesirably proliferating cell. Exemplary subjects include a subject having or at risk of having undesirable or aberrant cell proliferation; a subject having or at risk of having a benign hyperplasia; or a non-metastatic or metastatic neoplasia, cancer, tumor or malignancy (e.g., a solid or liquid tumor, myeloma, lymphoma, leukemia, carcinoma, sarcoma, melanoma, neural, reticuloendothelial and haematopoietic neoplasia).

In accordance with the invention, there are additionally provided methods of treating a subject having a hyperproliferative disorder and methods of treating a subject having a neoplasia, tumor, cancer or malignancy (metastatic, non-metastatic or benign). In various embodiments, a method includes, administering to a subject an amount of the drug conjugates sufficient to treat the hyperproliferative disorder; and administering to a subject an amount of the drug conjugates sufficient to reduce or inhibit proliferation of the neoplasia, tumor, cancer or malignancy.

Methods include treating a subject having or at risk of having a metastasis. For example, an amount of a drug conjugate effective to reduce or inhibit spread or dissemination of a tumor, cancer or neoplasia to other sites, locations or regions within the subject. In various embodiments, a method reduces or inhibits metastasis of a primary tumor or cancer to one or more other sites, formation or establishment of a metastasis at one or more other sites, locations or regions thereby reducing or inhibiting tumor or cancer relapse or tumor or cancer progression. In further embodiments, a method reduces or inhibits growth, proliferation, mobility or invasiveness of tumor or cancer cells that potentially do develop metastases (e.g., disseminated tumor cells); reduces or inhibits formation or establishment of metastases arising from a primary tumor or cancer to one or more other sites, locations or regions distinct from the primary tumor or cancer; reduces or inhibits growth or proliferation of a metastasis at one or more other sites, locations or regions distinct from the primary tumor or cancer after the metastasis has formed or has been established; or reduces or inhibits formation or establishment of additional metastasis after the metastasis has been formed or established. In yet another embodiment, a method reduces or inhibits relapse or progression of the neoplasia, tumor, cancer or malignancy.

In accordance with the invention, there are still further provided methods of reducing or inhibiting metastasis of a neoplasia, tumor, cancer or malignancy to other sites, or formation or establishment of metastatic neoplasia, tumor, cancer or malignancy at other sites distal from a primary neoplasia, tumor, cancer or malignancy. In various embodiments, a method includes administering to a subject an amount of the drug conjugate sufficient to reduce or inhibit metastasis of the neoplasia, tumor, cancer or malignancy to other sites, or formation or establishment of metastatic neoplasia, tumor, cancer or malignancy at other sites distal from the primary neoplasia, tumor, cancer or malignancy.

Neoplasia, tumor, cancer and malignancy treatable in accordance with the invention include solid cellular mass, hematopoietic cells, or a carcinoma, sarcoma (e.g. lymphosarcoma, liposarcoma, osteosarcoma, chondrosarcoma, leiomyosarcoma, rhabdomyosarcoma or fibrosarcoma), lymphoma, leukemia, adenoma, adenocarcinoma, melanoma, glioma, glioblastoma, meningioma, neuroblastoma, retinoblastoma, astrocytoma, oligodendrocytoma, mesothelioma, reticuloendothelial, lymphatic or haematopoietic (e.g., myeloma, lymphoma or leukemia) neoplasia, tumor, cancer or malignancy. Neoplasia, tumor, cancer and malignancy treatable in accordance with the invention can be present in or affect a lung (small cell lung or non-small cell lung cancer), thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, skin or stem cell neoplasia, tumor, cancer, or malignancy.

Methods may be practiced with other treatments or therapies (e.g., surgical resection, radiotherapy, ionizing or chemical radiation therapy, chemotherapy, immunotherapy, local or regional thermal (hyperthermia) therapy, or vaccination). Such treatments or therapies can be administered prior to, substantially contemporaneously with (separately or in a mixture), or following administration of a drug conjugate. In one embodiment, a method includes administering an anti-cell proliferative, anti-neoplastic, anti-tumor, anti-cancer or immune-enhancing treatment or therapy. In further embodiments, a method includes administering an alkylating agent, nucleoside or nucleotide analogs.

Cell or immunotherapies include a lymphocytes, plasma cells, macrophages, dendritic cells, T-cells, NK cells or B-cells; an antibody or small molecule targeting immune checkpoint inhibitors include PD1, TIGIT, LAG-3, TIM3, CTLA-4, etc, a cytokine or a chemokine (examples are interleukins IL-2, IFN-.gamma., IL-12).

Additional agents that are applicable with drug conjugates are targeted drugs or biological such as antibodies or small molecules. Non-limiting examples of monoclonal antibodies include rituximab (Rituxan®), trastuzumab (Herceptin), bevacizumab (Avastin), cetuximab (Erbitux), alemtuzumab (Campath), panitumumab (Vectibix), ibritumomab tiuxetan (Zevalin), tositumomab (Bexxar), etc. which can be used in combination with a drug conjugate in accordance with the invention. Other targeted drugs that are applicable for use with the drug conjugates are imatinib (Gleevec), gefitinib (Iressa), bortzomib (Velcade), lapatinib (Tykerb), sunitinib (Sutent), sorafenib (Nevaxar), nilotinib (Tasigna), etc.

Methods of treating a metastatic or non-metastatic tumor, cancer, malignancy or neoplasia, methods of treating a subject in need of treatment due to having or at risk of having a metastatic or non-metastatic tumor, cancer, malignancy or neoplasia, and methods of increasing effectiveness or improving an anti-proliferative, anti-tumor, anti-cancer, anti-neoplasia or anti-malignancy, therapy are provided. In respective embodiments, a method includes administering to a subject with or at risk of a metastatic or non-metastatic tumor, cancer, malignancy or neoplasia, an amount of a drug conjugate sufficient to treat the metastatic or non-metastatic tumor, cancer, malignancy or neoplasia; administering to the subject an amount of a drug conjugate sufficient to treat the subject; and administering to a subject that is undergoing or has undergone metastatic or non-metastatic tumor, cancer, malignancy or neoplasia therapy, an amount of a drug conjugate sufficient to increase effectiveness of the anti-proliferative, anti-tumor, anti-cancer, anti-neoplasia or anti-malignancy therapy.

Methods of the invention may be practiced prior to (i.e. prophylaxis), concurrently with or after evidence of the presence of undesirable or aberrant cell proliferation or a hyperproliferative disorder, disease or condition begins (e.g., one or more symptoms). Administering a drug conjugate prior to, concurrently with or immediately following development of a symptom of undesirable or aberrant cell proliferation or a hyperproliferative disorder may decrease the occurrence, frequency, severity, progression, or duration of one or more symptoms of the undesirable or aberrant cell proliferation or a hyperproliferative disorder, disease or condition in the subject. In addition, administering a drug conjugate prior to, concurrently with or immediately following development of one or more symptoms of the undesirable or aberrant cell proliferation or a hyperproliferative disorder, disease or condition may inhibit, decrease or prevent the spread or dissemination of hyperproliferating cells (e.g., metastasis) to other sites, regions, tissues or organs in a subject, or establishment of hyperproliferating cells (e.g., metastasis) at other sites, regions, tissues or organs in a subject.

Drug conjugates and the methods of the invention, such as treatment methods, can provide a detectable or measurable therapeutic benefit or improvement to a subject. A therapeutic benefit or improvement is any measurable or detectable, objective or subjective, transient, temporary, or longer-term benefit to the subject or improvement in the condition, disorder or disease, an adverse symptom, consequence or underlying cause, of any degree, in a tissue, organ, cell or cell population of the subject. Therapeutic benefits and improvements include, but are not limited to, reducing or decreasing occurrence, frequency, severity, progression, or duration of one or more symptoms or complications associated with a disorder, disease or condition, or an underlying cause or consequential effect of the disorder, disease or condition. Drug conjugates and methods of the invention therefore include providing a therapeutic benefit or improvement to a subject.

In a method of the invention in which a therapeutic benefit or improvement is a desired outcome, a drug conjugate of the invention can be administered in a sufficient or effective amount to a subject in need thereof. An “amount sufficient” or “amount effective” refers to an amount that provides, in single or multiple doses, alone or in combination, with one or more other compositions (therapeutic agents such as a chemotherapeutic or immune stimulating drug), treatments, protocols, or therapeutic regimens agents, a detectable response of any duration of time (long or short term), a desired outcome in or a benefit to a subject of any measurable or detectable degree or for any duration of time (e.g., for hours, days, months, years, or cured). The doses or “sufficient amount” or “effective amount” for treatment (e.g., to provide a therapeutic benefit or improvement) typically are effective to ameliorate a disorder, disease or condition, or one, multiple or all adverse symptoms, consequences or complications of the disorder, disease or condition, to a measurable extent, although reducing or inhibiting a progression or worsening of the disorder, disease or condition or a symptom, is considered a satisfactory outcome.

The term “ameliorate” means a detectable objective or subjective improvement in a subject's condition. A detectable improvement includes a subjective or objective reduction in the occurrence, frequency, severity, progression, or duration of a symptom caused by or associated with a disorder, disease or condition, an improvement in an underlying cause or a consequence of the disorder, disease or condition, or a reversal of the disorder, disease or condition.

Treatment can therefore result in inhibiting, reducing or preventing a disorder, disease or condition, or an associated symptom or consequence, or underlying cause; inhibiting, reducing or preventing a progression or worsening of a disorder, disease, condition, symptom or consequence, or underlying cause; or further deterioration or occurrence of one or more additional symptoms of the disorder, disease condition, or symptom. Thus, a successful treatment outcome leads to a “therapeutic effect,” or “benefit” or inhibiting, reducing or preventing the occurrence, frequency, severity, progression, or duration of one or more symptoms or underlying causes or consequences of a condition, disorder, disease or symptom in the subject. Treatment methods affecting one or more underlying causes of the condition, disorder, disease or symptom are therefore considered to be beneficial. Stabilizing or inhibiting progression or worsening of a disorder or condition is also a successful treatment outcome.

A therapeutic benefit or improvement therefore need not be complete ablation of any one, most or all symptoms, complications, consequences or underlying causes associated with the condition, disorder or disease. Thus, a satisfactory endpoint is achieved when there is an incremental improvement in a subject's condition, or a partial reduction in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of one or more of the physiological, biochemical or cellular manifestations or characteristics of the disorder or disease, over a short or long duration of time (hours, days, weeks, months, etc.).

In particular embodiments, a method of treatment results in partial or complete destruction of a metastatic or non-metastatic tumor, cancer, malignant or neoplastic cell mass, volume, size or numbers of cells; results in stimulating, inducing or increasing necrosis, lysis or apoptosis of metastatic or non-metastatic tumor, cancer, malignant or neoplastic cell; results in reducing metastatic or non-metastatic tumor, cancer, malignant or neoplastic volume, size, cell mass; results in inhibiting or preventing progression or an increase in metastatic or non-metastatic tumor, cancer, malignant or neoplastic volume, mass, size or cell numbers; results in inhibiting or decreasing the spread or dissemination of hyperproliferating cells (e.g., metastasis) to other (secondary) sites, regions, tissues or organs in a subject, or establishment of hyperproliferating cells (e.g., metastasis) at other (secondary) sites, regions, tissues or organs in a subject; or results in prolonging lifespan of the subject. In additional particular embodiments, a method of treatment results in reducing or decreasing severity, duration or frequency of an adverse symptom or complication associated with or caused by the metastatic or non-metastatic tumor, cancer, malignancy or neoplasia.

An amount sufficient or an amount effective can but need not be provided in a single administration and, can but need not be, administered alone or in combination with another composition (e.g., chemotherapeutic or immune enhancing or stimulating agent), treatment, protocol or therapeutic regimen. For example, the amount may be proportionally increased as indicated by the need of the subject, status of the disorder, disease or condition treated or the side effects of treatment. In addition, an amount sufficient or an amount effective need not be sufficient or effective if given in single or multiple doses without a second composition (e.g., chemotherapeutic or immune stimulating agent), treatment, protocol or therapeutic regimen, since additional doses, amounts or duration above and beyond such doses, or additional compositions (e.g., chemotherapeutic or immune stimulating agents), treatments, protocols or therapeutic regimens may be included in order to be considered effective or sufficient in a given subject. Amounts considered sufficient also include amounts that result in a reduction of the use of another treatment, therapeutic regimen or protocol.

An amount sufficient or an amount effective need not be effective in each and every subject treated, prophylactically or therapeutically, nor a majority of treated subjects in a given group or population. As is typical for treatment or therapeutic methods, some subjects will exhibit greater or less response to a given treatment, therapeutic regimen or protocol. An amount sufficient or an amount effective refers to sufficiency or effectiveness in a particular subject, not a group or the general population. Such amounts will depend in part upon the condition treated, such as the type or stage of undesirable or aberrant cell proliferation or hyperproliferative disorder (e.g., a metastatic or non-metastatic tumor, cancer, malignancy or neoplasia), the therapeutic effect desired, as well as the individual subject (e.g., the bioavailability within the subject, gender, age, etc.).

Particular non-limiting examples of therapeutic benefit or improvement for undesirable or aberrant cell proliferation, such as a hyperproliferative disorder (e.g., a metastatic or non-metastatic tumor, cancer, malignancy or neoplasia) include a reduction in cell size, mass or volume, inhibiting an increase in cell size, mass or volume, a slowing or inhibition of worsening or progression, stimulating cell necrosis, lysis or apoptosis, reducing or inhibiting neoplastic or tumor malignancy or metastasis, reducing mortality, and prolonging lifespan of a subject. Thus, inhibiting or delaying an increase in cell size, mass, volume or metastasis (stabilization) can increase lifespan (reduce mortality) even if only for a few days, weeks or months, even though complete ablation of the metastatic or non-metastatic tumor, cancer, malignancy or neoplasia has not occurred. Adverse symptoms and complications associated with a hyperproliferative disorder (e.g., a metastatic or non-metastatic tumor, cancer, malignancy or neoplasia) that can be reduced or decreased include, for example, pain, nausea, discomfort, lack of appetite, lethargy and weakness. A reduction in the occurrence, frequency, severity, progression, or duration of a symptom of undesirable or aberrant cell proliferation, such as a hyperproliferative disorder (e.g., a metastatic or non-metastatic tumor, cancer, malignancy or neoplasia), such as an improvement in subjective feeling (e.g., increased energy, appetite, reduced nausea, improved mobility or psychological well being, etc.), are therefore all examples of therapeutic benefit or improvement.

For example, a sufficient or effective amount of a drug conjugate is considered as having a therapeutic effect if administration results in less chemotherapeutic drug, radiation or immunotherapy being required for treatment of undesirable or aberrant cell proliferation, such as a hyperproliferative disorder (e.g., a metastatic or non-metastatic tumor, cancer, malignancy or neoplasia).

The term “subject” refers to animals, typically mammalian animals, such as humans, non human primates (apes, gibbons, chimpanzees, orangutans, macaques), domestic animals (dogs and cats), farm animals (horses, cows, goats, sheep, pigs) and experimental animal (mouse, rat, rabbit, guinea pig). Subjects include animal disease models, for example, animal models of undesirable or aberrant cell proliferation, such as a hyperproliferative disorder (e.g., a metastatic or non-metastatic tumor, cancer, malignancy or neoplasia) for analysis of drug conjugate in vivo.

Subjects appropriate for treatment include those having or at risk of having a metastatic or non-metastatic tumor, cancer, malignant or neoplastic cell, those undergoing as well as those who are undergoing or have undergone anti-proliferative (e.g., metastatic or non-metastatic tumor, cancer, malignancy or neoplasia) therapy, including subjects where the tumor is in remission. “At risk” subjects typically have risk factors associated with undesirable or aberrant cell proliferation, development of hyperplasia (e.g., a tumor).

Particular examples of at risk or candidate subjects include those with cells that express a receptor to which the drug conjugate can bind, particularly where cells targeted for inhibition of proliferation or induction of apoptosis express greater numbers or amounts of receptor than non-target cells. Such cells can be selectively or preferentially targeted for inhibition of proliferation or induction of apoptosis.

At risk subjects also include those that are candidates for and those that have undergone surgical resection, chemotherapy, immunotherapy, ionizing or chemical radiotherapy, local or regional thermal (hyperthermia) therapy, or vaccination. The invention is therefore applicable to treating a subject who is at risk of a metastatic or non-metastatic tumor, cancer, malignancy or neoplasia or a complication associated with a metastatic or non-metastatic tumor, cancer, malignancy or neoplasia, for example, due to metastatic or non-metastatic tumor, cancer, malignancy or neoplasia reappearance or regrowth following a period of stability or remission.

Risk factors include gender, lifestyle (diet, smoking), occupation (medical and clinical personnel, agricultural and livestock workers), environmental factors (carcinogen exposure), family history (autoimmune disorders, diabetes, etc.), genetic predisposition, etc. For example, subjects at risk for developing melanoma include excess sun exposure (ultraviolet radiation), fair skin, high numbers of naevi (dysplastic nevus), patient phenotype, family history, or a history of a previous melanoma. Subjects at risk for developing cancer can therefore be identified by lifestyle, occupation, environmental factors, family history, and genetic screens for tumor associated genes, gene deletions or gene mutations.

Subjects also include those precluded from other treatments. For example, certain subjects may not be good candidates for surgical resection, chemotherapy, immunotherapy, ionizing or chemical radiotherapy, local or regional thermal (hyperthermia) therapy, or vaccination. Thus, candidate subjects for treatment in accordance with the invention include those that are not a candidate for surgical resection, chemotherapy, immunotherapy, ionizing or chemical radiotherapy, local or regional thermal (hyperthermia) therapy, or vaccination.

Drug conjugates may be formulated in a unit dose or unit dosage form. In a particular embodiment, a drug conjugate is in an amount effective to treat a subject having undesirable or aberrant cell proliferation or a hyperproliferative disorder. In an additional particular embodiment, a drug conjugate is in an amount effective to treat a subject having a metastatic or non-metastatic tumor, cancer, malignancy or neoplasia.

Compositions and methods of the invention may be contacted or provided in vitro, ex vivo or in vivo. Compositions can be administered to provide the intended effect as a single or multiple dosages, for example, in an effective or sufficient amount. Exemplary doses range from about 0.1-20 mg/kg.

Compositions can be administered and methods may be practiced via systemic, regional or local administration, by any route.

The invention further provides drug conjugates and methods wherein the drug conjugates are included in pharmaceutical compositions. A pharmaceutical composition refers to “pharmaceutically acceptable” and “physiologically acceptable” carriers, diluents or excipients. As used herein, the term “pharmaceutically acceptable” and “physiologically acceptable,” when referring to carriers, diluents or excipients includes solvents (aqueous or non-aqueous), detergents, solutions, emulsions, dispersion media, coatings, isotonic and absorption promoting or delaying agents, compatible with pharmaceutical administration and with the other components of the formulation.

Pharmaceutical compositions can be formulated in a sterile diluent, such as saline solution, fixed oils, polyethylene glycols, glycerine, propylene glycol or other synthetic solvents. The preparation may contain one or more preservatives to prevent microorganism growth (e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose).

Pharmaceutical compositions for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and polyetheylene glycol), and suitable mixtures thereof. Fluidity can be maintained, for example, by the use of a coating such as lecithin, or by the use of surfactants. Antibacterial and antifungal agents include, for example, parabens, chlorobutanol, phenol, ascorbic acid and thimerosal. Including an agent that delays absorption, for example, aluminum monostearate and gelatin can prolonged absorption of injectable compositions.

Additional pharmaceutical formulations and delivery systems are known in the art and are applicable in the methods of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms, Technonic Publishing Co., Inc., Lancaster, Pa., (1993); and Poznansky, et al., Drug Delivery Systems, R. L. Juliano, ed., Oxford, N.Y. (1980), pp. 253-315).

The invention provides kits including drug conjugates of the invention, combination compositions and pharmaceutical formulations thereof, packaged into suitable packaging material. A kit optionally includes a label or packaging insert including a description of the components or instructions for use in vivo, or ex vivo, of the components therein. Exemplary instructions include instructions for reducing or inhibiting proliferation of a cell, reducing or inhibiting proliferation of undesirable or aberrant cells, such as a hyperproliferating cell, reducing or inhibiting proliferation of a metastatic or non-metastatic tumor, cancer, malignant or neoplastic cell, treating a subject having a hyperproliferative disorder, treating a subject having a metastatic or non-metastatic tumor, cancer, malignancy or neoplasia, or reducing fertility of an animal.

A kit can contain a collection of such components, e.g., two or more drug conjugates alone, or in combination with another therapeutically useful composition (e.g., an anti-proliferative or immune-enhancing drug).

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.). Kits of the invention can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., floppy diskette, hard disk, ZIP disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacturer location and date.

Labels or inserts can include information on a condition, disorder, disease or symptom for which a kit component may be used. Labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Exemplary instructions include, instructions for treating an undesirable or aberrant cell proliferation, hyperproliferating cells and disorders (e.g., metastatic or non-metastatic tumor, cancer, malignancy or neoplasia). Kits of the invention therefore can additionally include labels or instructions for practicing any of the methods of the invention described herein including treatment methods.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

As used herein, the singular forms “a”, “and, “the” include plural referents unless the context clearly indicates otherwise. Thus, for example, reference to “a drug conjugate” includes a plurality of such drug conjugates.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### BRIEF DESCRIPTION OF THE DRAWINGS

FIG. 1A depicts the structure of LHRH-I drug conjugate OTS-P22 in accordance with a preferred embodiment of the present invention;

FIG. 1B depicts the RP-HPLC analysis of LHRH-I drug conjugate OTS-P22 in accordance with a preferred embodiment of the present invention;

FIG. 1C depicts the mass spectrometric analysis of LHRH-I drug conjugate OTS-P22 in accordance with a preferred embodiment of the present invention.

FIG. 2A depicts the effect of drug conjugate OTS-P22 on the viability of HCC1806, and BT-549 TNBC cell lines as well as on normal 3T3 mouse fibroblasts in an MTS proliferation assay in accordance with the preferred embodiment of the present invention;

FIG. 2B depicts the effect of OTS-P22 on caspase activity in HCC1806 cells in accordance with the preferred embodiment of the present invention;

FIG. 3A, 3B depicts the LHRH receptor mRNA expression in 3T3 normal fibroblasts, BT-20, BT-549 and HCC1806 TNBC cell lines as well as Chem-1 overexpressing LHRH receptor (positive control) and PDX1 (BR1367), PDX2 (J000080739F) and PDX3 (BR1126) patient-derived tumors.

FIG. 4A depicts the effect of saline, OTS-P22 and unconjugated MMAE on tumor growth of HCC1806 xenografted into female beige SCID mice in accordance with the preferred embodiment of the present invention;

FIG. 4B depicts animal weight of saline-, OTS-P22- or unconjugated MMAE-treated female beige SCID mice bearing HCC1806 xenografts into in accordance with the preferred embodiment of the present invention;

FIG. 5A depict hematology parameters of saline- and OTS-P22 treated SCID mice bearing HCC1806 TNBC xenografts into in accordance with the preferred embodiment of the present invention;

FIG. 5B depict hematoxylin-eosin staining of normal organs from saline- and OTS-P22 treated SCID mice bearing HCC1806 TNBC xenografts into in accordance with the preferred embodiment of the present invention;

FIG. 6A depicts RP-HPLC analysis of drug conjugate OTS-P23 in accordance with a preferred embodiment of the present invention;

FIG. 6B depicts mass spectrometric analysis of drug conjugate OTS-P23 in accordance with a preferred embodiment of the present invention;

FIGS. 7A, 7B, 7C and 7D depicts the stability of OTS-P23 in human plasma at 0, 2, 8 and 24 hours incubation times respectively in accordance with a preferred embodiment of the present invention;

Table 1 depicts competition binding IC₅₀ of [DTrp⁶]LHRH, OTS-P22 and OTS-P23 with ¹²⁵I[DTrp⁶]LHRH to membranes from Chem-1 cells over-expressing LHRH receptor in accordance with a preferred embodiment of the present invention; table 1 in in drawings folder.

FIG. 8A depicts the effect of drug conjugate OTS-P23 on the viability of HCC1806 and normal 3T3 fibroblasts in an MTS proliferation assay in accordance with the preferred embodiment of the present invention;

FIG. 8B depicts the effect of OTS-P23 on caspase activity in HCC1806 cells in accordance with the preferred embodiment of the present invention;

FIG. 9A depicts RP-HPLC analysis of drug conjugate OTS-P23b in accordance with a preferred embodiment of the present invention;

FIG. 9B depicts mass spectrometric analysis of drug conjugate OTS-P23b in accordance with a preferred embodiment of the present invention;

FIG. 10A depicts RP-HPLC analysis of drug conjugate OTS-P26 in accordance with a preferred embodiment of the present invention;

FIG. 10B depicts mass spectrometric analysis of drug conjugate OTS-P26 in accordance with a preferred embodiment of the present invention;

FIG. 11 depicts the effect of drug conjugate OTS-P26 on the viability of HCC1806 TNBC cells in an MTS proliferation assay in accordance with the preferred embodiment of the present invention;

FIG. 12A depicts RP-HPLC analysis of drug conjugate OTS-P28 in accordance with a preferred embodiment of the present invention;

FIG. 12B depicts mass spectrometric analysis of drug conjugate OTS-P28 in accordance with a preferred embodiment of the present invention;

FIGS. 13A, 13B, 13C and 13D depicts the stability of OTS-P28 in human plasma at 0, 2, 8 and 24 hours incubation times respectively in accordance with a preferred embodiment of the present invention;

FIG. 14A depicts RP-HPLC analysis of drug conjugate OTS-P31 in accordance with a preferred embodiment of the present invention;

FIG. 14B depicts mass spectrometric analysis of drug conjugate OTS-P31 in accordance with a preferred embodiment of the present invention;

FIG. 15 depicts schematic of LHRHa carrying two copies of Val-Cit-PABC-MMAE via DBCO-N-bis(PEG₄-NHS ester) branched spacer.

FIG. 16 depicts schematic of LHRHa carrying one copy of Val-Cit-PABC-MMAE and one copy of Val-Cit-PABC-DMEA-seco-DUBA via DBCO-N-bis(PEG₄-NHS ester) branched spacer.

FIG. 17 depicts schematic of dimeric LHRHa carrying two copies of LHRHa and one copy of Val-Cit-PABC-MMAE via DBCO-N-bis(PEG₄-NHS ester) branched spacer.

### DETAILED DESCRIPTION OF THE PREFERRED EMBODIMENT

In the following discussion that addresses a number of embodiments and applications of the present invention, reference is made to the accompanying drawings that form a part hereof, and show by way of illustration specific embodiments in which the invention may be practiced. It is to be understood that other embodiments may be utilized and changes may be made without departing from the scope of the present invention.

Various inventive features are described below that can be used independently of one another or in combination with other features. Any single inventive feature may not address any of the problems discussed above or only address one of the problems discussed above. Further, one or more of the problems discussed above may not be fully addressed by any of the features described below.

As used herein, the singular forms “a”, “an” and “the” include plural referents unless the context clearly dictates otherwise. “And” as used herein is interchangeably used with “or” unless expressly stated otherwise. All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively.

Additionally, the words “herein,” “wherein”, “whereas”, “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

The present invention provides compounds and methods for generating new highly stable monomeric LHRHa conjugates carrying highly potent payloads (by way of example MMAE) which will allow for their effective delivery to treat tumor cells with decreased adverse side effects associated with existing drugs, and limit nonselective activity. In a preferred embodiment, LHRH-I analogs with functional groups such as [D-Lys^{6 (N3)}], [D-Lys⁶], [D-Lys^{6 (Aoa)}], D-Cys⁶] or activated [D-Glu⁶] or [D-Asp⁶] are conjugated to DBCO-spacer-linker-payload, NHS ester-spacer-linker-payload, ketone- or aldehyde-spacer-linker-payload, Mal-spacer-linker-payload or NH₂-spacer-linker-payload respectively generating triazole, amide, oxime, thioether or amide bonds respectively. The attachment of the spacer-linker-payload on position 6 of the LHRH analogs ensures that the intrinsic properties of the LHRH peptide are maintained. In the preferred embodiment, highly stable triazole, thioether or amide bonds are formed between the components of the conjugate. Existing published reports have shown that such conjugates are cleaved at the peptidic linker by cathepsin B which is highly present in the lysosomes of cancer cells. The cleaved MMAE then exits the endosomes into the intracellular department and bind to microtubules blocking their polymerization and thus inhibiting cell proliferation and inducing apoptosis. Similarly, drug conjugates could also be generated using LHRH-II or LHRH-III analogs with functional groups such as [D-Lys^{4 (N3)}], [D-Lys⁴], [D-Lys-Aoa⁴], [D-Cys⁴], activated [D-Glu⁴] or [D-Asp⁴], [D-Lys^{8(N3)}], [D-Lys⁸], D-Lys-Aoa⁸,[D-Cys⁸], activated [D-Glu⁸] or [D-Asp⁸] which are conjugated to DBCO-spacer-linker-payload, NHS ester-spacer-linker-payload, Mal-spacer-linker-payload, Aldehyde-spacer-linker-payload, Ketone-spacer-linker-payload or NH₂-spacer-linker-payload respectively. The spacer could be linear or branched. The branched spacer could carry monomeric or multimeric LHRH analogs. The conjugate could also carry a single or multiple copies of the same payload or different payloads. For example a conjugate carrying 1, 2 or 3 copies of MMAE (tubulin polymerization inhibitor) or a conjugate carrying one copy of MMAE and one copy of seco-DUBA (DNA minor groove alkylating agent) or a conjugate carrying one copy of MMAE, one copy of seco-DUBA and one copy of Exatecan (Topoisomerase I inhibitor).

In the preferred embodiment, monomeric [D-Lys^{6(N3)}]LHRHa is conjugated to DBCO-dPEG₃-Val-Cit-PABC-MMAE to generate OTS-P22 or to DBCO-dPEGg-Val-Cit-PABC-MMAE to generate OTS-P23 or to DBCO-dPEG₅-Val-Cit-PABC-MMAE to generate OTS-P23b. Similarly [D-Cys⁶]LHRHa is conjugated to MC-Val-Cit-PABC-MMAE to generate OTS-P26 or to Mal-dPEGg-Val-Cit-PABC-MMAE to generate OTS-P28. Similarly [D-Lys⁶]LHRHa is conjugated to NHS ester-Glut-Val-Cit-PABC-MMAE to generate OTS-P31.

In one embodiment, MMAE may be substituted with any other highly potent cytotoxic agents including but not limited to MMAF, MMAD, tubulysin, DM1 (Mertansine), PNU-159682 (doxorubicin metabolite), Duocarmycins, seco-DUBA, PBD (Pyrrolobenzodiazepine), SG3199 (Pyrrolobenzodiazepine dimer), DX8951 (Exatecan).

In one embodiment, peptidic linkers other than the Val-Cit-PABC may be used, either alone or in combination with other linkers.

Finally, in yet another embodiment, the targeting peptide can be endogenous LHRH or an analog thereof, including but not limited to LHRH receptor agonists and antagonists. See A. V. Schally, et al., “Peptide analogs in the therapy of prostate cancer,” Prostate, 45, pp. 158-66 (2000); W. R. Miller, “Growth of human breast cancer cells inhibited by a luteinizing hormone-releasing hormone agonist,” Nature, 313, pp. 231-33 (1985); K. Szepashzi, et al., “Effective treatment of advanced estrogen-independent MXT mouse mammary cancers with targeted cytotoxic LH-RH analogs,” Breast Cancer Res. Treat., 56, pp. 267-76 (1999).

The novel LHRHa-MMAE conjugates are intended to be used in the treatment of reproductive and non-reproductive cancers expressing the LHRH receptor. The use of the novel LHRHa-MMAE conjugates will increase the concentration of MMAE anticancer drug in tumor cells and attenuate unnecessary exposure to normal cells. The novel LHRHa-MMAE conjugates have applications to prostate cancer and other metastatic cancers such as breast, ovarian, endometrial, kidney, cervical, lung and pancreatic cancers as well as other cancers which express LHRH receptors, especially in late stage, highly invasive and aggressive stage IV tumors, and in reoccurring tumors as these are the most difficult to treat. The novel LHRHa-MMAE conjugates have also applications to brain tumors including glioblastomas.

In one embodiment the novel LHRHa-MMAE conjugates targets and binds to the LHRH receptor of any cancer cell or any tumor microenvironment that expresses these receptors. Moreover, protease produced by the tumor cells effectively cleave the linker to release the “warhead,” i.e., MMAE or the above mentioned payloads, and thereby to achieve more effective concentrations of the therapeutic agents in the targeted area. Further, the targeted novel LHRHa-MMAE conjugates will selectively kill tumor cells expressing LHRH receptor (direct killing) or through bystander effect preventing or reducing dose-limiting systemic toxicity of said chemotherapeutic drugs.

LHRHa-MMAE conjugates are projected to be used in clinical settings to target cancers expressing the LHRH receptor in both animals and humans.

A compound that is a “conjugate” of two domains refers to a compound in which the two domains (or moieties) are covalently bonded to one another, either directly or via a spacer or a linker. Compounds used in the present invention may be administered to a patient by any “effective route”. Said effective route may include intravenous injection or infusion as well as direct injection to tumor sites.

Compounds used in the present invention may be administered by any pharmaceutically acceptable carrier preparations. The carriers and preparations may include sterile, aqueous or non-aqueous solutions, suspensions, and emulsions. Compounds may be administered in combination with a slow-release mechanism. Examples of non-aqueous solvents are propylene glycol, polyethylene glycol, vegetable oils, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The active therapeutic ingredient may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as antimicrobials, anti-oxidants, chelating agents, inert gases, and the like.

The form may vary depending upon the route of administration. For example, compositions for injection may be provided in the form of an ampoule, each containing a unit dose amount, or in the form of a container containing multiple doses.

A compound in accordance with the present invention may be formulated into therapeutic compositions as pharmaceutically acceptable salts. These salts include those formed from inorganic bases such as sodium, potassium, ammonium, calcium or ferric hydroxides, and organic bases such as isopropylamine, trimethylamine, histidine, procaine and the like.

As used herein, an “effective amount” of a compound is an amount that when administered to a patient inhibits or reduces the growth of targeted tumors to a clinically significant degree; or alternatively, to a statistically significant degree as compared to control.

As used herein, a “MMAE drug” is monomethyl auristatin E which blocks tubulin polymerization and causes inhibition of cell proliferation and induction of apoptosis.

As used herein, a “spacer” refers to any spacers known in the art including linear or branched PEG.

As used herein, a “linker” refers to any peptide linker known in the art including valine-Citrulline-PABC, Val-Ala, Gly-Gly-Phe-Gly or other peptide linkers.

As used herein, an “analog” is a molecule comprised of a peptide sequence that is comparable to the endogenously produced compound, in which, one or more residues have been replaced, deleted or modified with an alternate desired moiety. An analog of LHRH (I, II, III) can be a natural or a synthetic peptide that resembles LHRH (I, II, III) in structure and/or function.

All the references cited in this disclosure are hereby incorporated by reference in their entirety.

The foregoing description of several embodiments of the LHRHa-MMAE conjugates and treatment methods utilizing the LHRHa-MMAE conjugates has been presented for purposes of illustration. It is not intended to be exhaustive or to limit the application to the precise forms disclosed, and obviously, many modifications and variations are possible in light of the above teaching. It is understood that the invention may be applied in ways other than as specifically set forth herein without departing from the scope of the invention.

### EXAMPLES

The following examples illustrate the innovation and significance of using monomeric, multimeric LHRHa conjugates. In addition, conjugates could carry two or multiple copies of the same payload or different payloads are presented. Examples 1-14 are for LHRHa-based conjugates carrying MMAE (tubulin polymerization inhibitor) using studies involving triple-negative breast cancer, however, the applications of, mechanisms of action and/or relevance of MMAE-LHRHa conjugates are not restrictive to treatment of those cancers. Similarly, MMAE could be substituted by other highly potent payloads including but not limited to MMAF, MMAD, tubulysin, DM1 (Mertansine), PNU-159682 (doxorubicin metabolite), Duocarmycins, seo-DUBA, PBD (Pyrrolobenzodiazepine), SG3199 (Pyrrolobenzodiazepine dimer), DX8951 or other highly potent payloads. In addition, linear or branched spacers could carry different functional reactive groups than NHS ester and DBCO. Furthermore, branched spacers could have more than 3 arms with similar or different reactive functional groups.

### Example 1

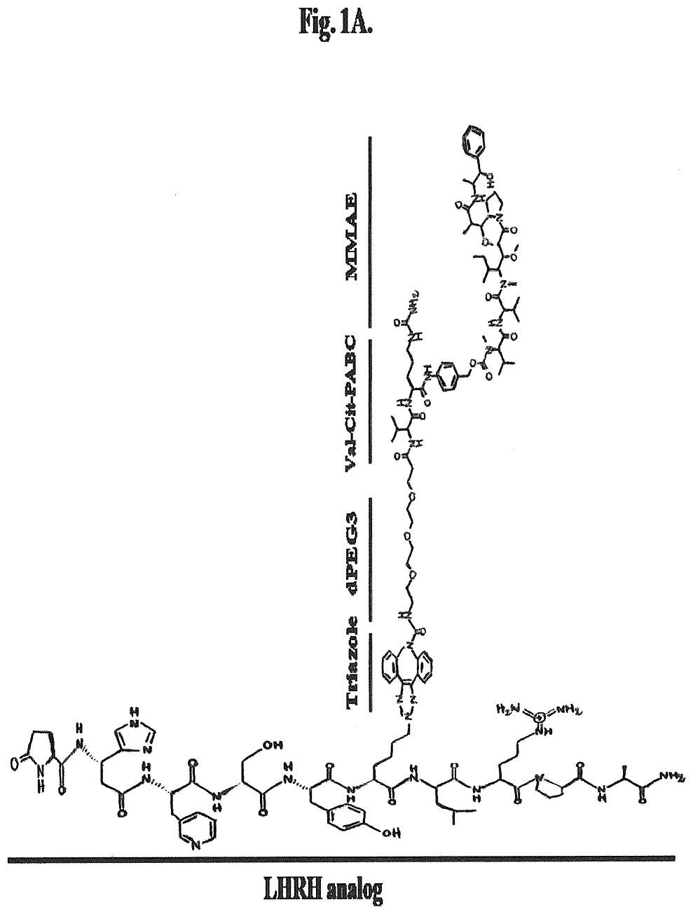

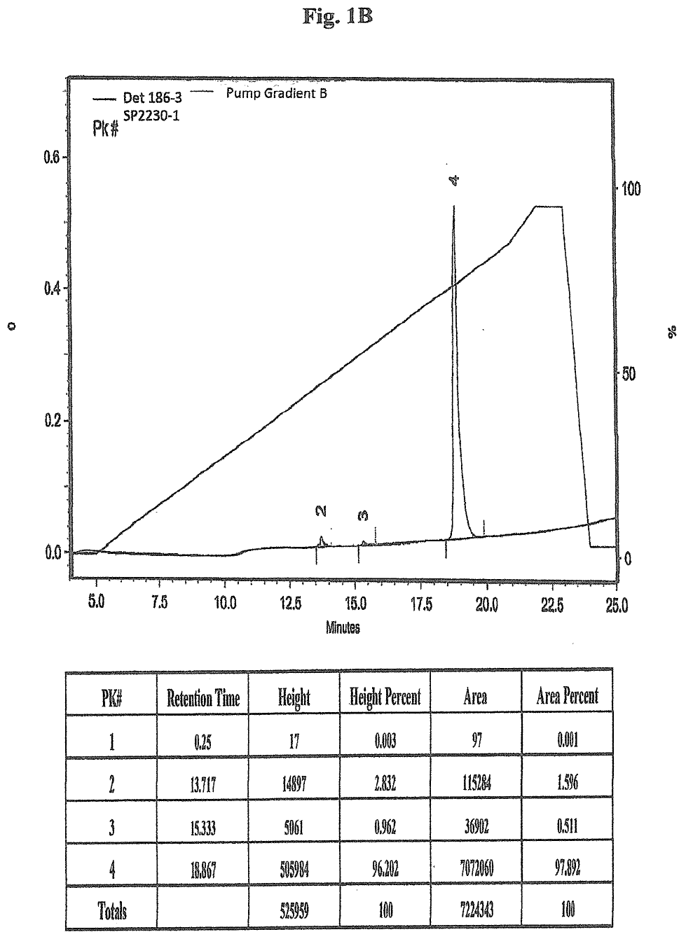

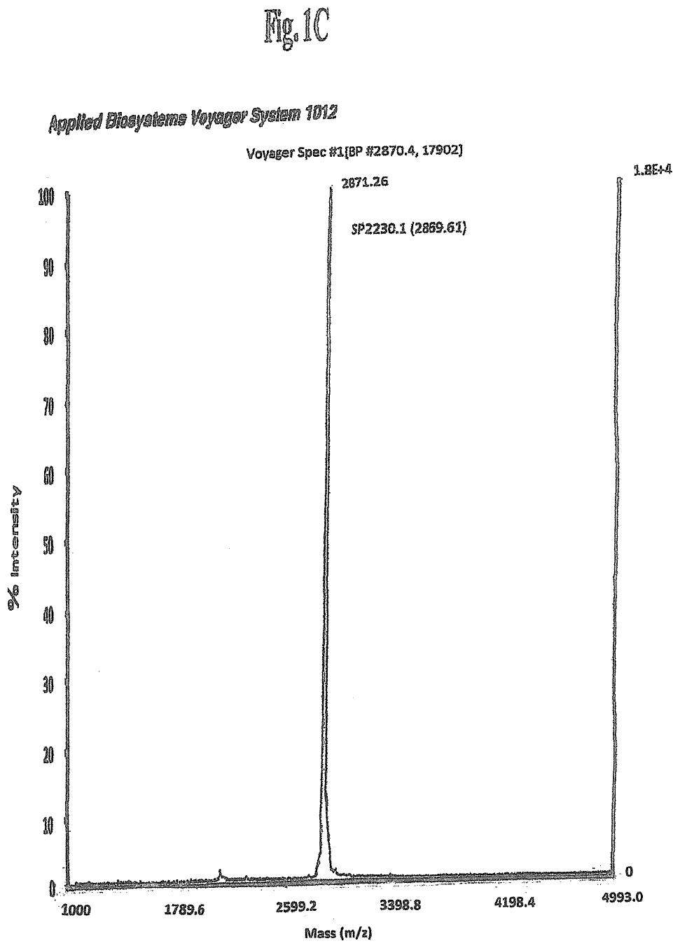

FIG. 1A depicts the structure of LHRHa-based drug conjugate OTS-P22 in accordance with a preferred embodiment of the present invention;

FIG. 1B depicts the RP-HPLC analysis of LHRH drug conjugate OTS-P22. The synthesis of the ligand Glp-His-DPal-Ser-Tyr-DLys(azide)-Leu-Arg-Pro-DAla-amide and DBCO-dPEG₃-Val-Cit-PABC-MMAE was accomplished using Fmoc or Boc solid phase peptide synthesis (SPPS). After chain assembly, peptides are cleaved from the solid support and purified to homogeneity by RP-HPLC as described below. The ligand was then conjugated through its DLysine (azide) to DBCO-dPEG₃-Val-Cit-PABC-MMAE at 1/1.2 molar ratio in DMF at room temperature overnight by means of copper-free click chemistry and generating drug conjugate OTS-P22 with the structure Glp-His-DPal-Ser-Tyr-DLys[dPEG₃-Val-Cit-PABC-MMAE]-Leu-Arg-Pro-DAla-amide. The conjugation of DBCO to azide group generated a stable triazole bond. OTS-P22 was then purified using C18 Agilent 1100 instrument (4.6 mm×250 mm, 5 micron) at a flow rate of 1.2 ml/min in presence of buffer A (0.1% TFA in water) and buffer B (0.1% TFA in acetonitrile) at linear gradient (20%-40%) of buffer B during 20 minutes run time according to the protocol described in FIG. 1. OTS-P22 was eluted at minute 18.867 at 97.9% purity.

FIG. 1C depicts the mass spectrometric analysis (MS) of drug conjugate OTS-P22 using Applied Biosystems Voyager System 1099 at 0.95 min run time and 1 ml/min flow rate. OTS-P22 has molecular weight of 2869.61 dalton.

### Example 2

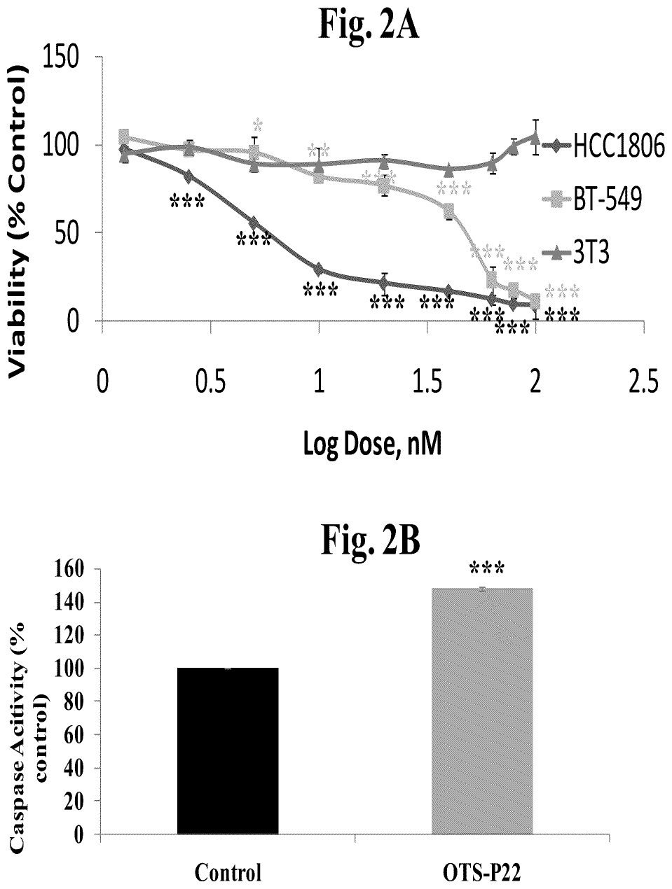

FIG. 2A depicts the viability of HCC1806 and BT-549 TNBC cell lines as well as normal 3T3 mouse fibroblasts in presence of peptide drug conjugate OTS-P22 at increasing concentrations (Log 0.165-100 nM). Cells were plated at 10,000 cells/well on 96-well plates in RPMI or DMEM medium (pH 7.4) with 10% FBS or 10% BCS and after attachment they were washed and incubated with OTS-P22 in complete medium without antibiotics for 3 hours. After 3 hours, cells were washed and then incubated in complete medium without antibiotics for an additional 93 hours. At the end of the incubation period cells were washed and cell viability was measured using MTS assay according to the manufacturer's instructions (Promega, Madison, WI). Data shown are percentage mean value and standard error of triplicate measurements. Differences between mean values were evaluated by one-way ANOVA followed by Tukey's honestly significant difference procedure. **<0.01 were highly significant. We found OTS-P22 to be highly potent in reducing the viability of HCC1806 (IC₅₀: 6.8 nM) and BT-549 (IC₅₀: 35.4 nM) TNBC cell lines with no effect on 3T3 normal fibroblasts.

FIG. 2B depicts the caspase activity in HCC1806 TNBC cell lines treated with saline or OTS-P22. Briefly, HCC1806 cells were plated at 10⁶ cells per T35 flask and after attachment they were washed then incubated for 48 hours with PBS or 100 nM of OTS-P22. At the end of the incubation, cells were washed three times, lyzed and proteins quantified using Bradford reagents. Apoptosis assay was performed using 100 μg proteins according to ApoTarget Caspase Colorimetric Protease Assay Sampler Kit instructions (Invitrogen, Carlsbad, CA). Data are presented as percentage of control (PBS) and statistical analysis was performed by one-way ANOVA test followed by Tukey's honestly significant difference procedure. ***<0.001 were highly significant. OTS-P22 significantly increased caspase activity by 1.5 fold in HCC1806 TNBC cells as compared to saline-treated cells (*** p<0.001).

### Example 3

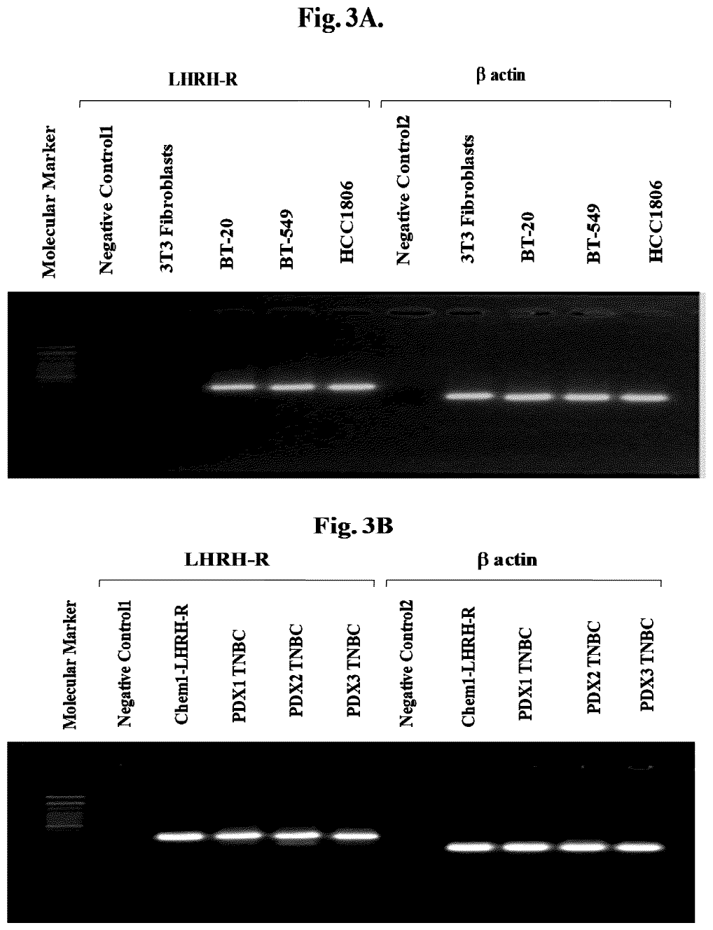

FIG. 3A, 3B depicts the One-step RT-PCR analysis of mRNA for LHRH-R and β-actin using 200 ng of total RNA. The PCR products containing SmartGlow™ loading buffer were separated by 1.8% agarose gel electrophoresis and visualized with SmartBlue transilluminator (E4000). 3A. PCR products from negative control, 3T3, BT-20, BT-549 and HCC1806 TNBC cell lines. 3B. PCR products from Chem-1 LHRH-R overexpressing cells (positive control), PDX1 (BR1367), PDX2 (J000080739F) and PDX3 (BR1126). The PCR products obtained were of expected size of 219 bp (LHRH-R) and 184 bp (β-actin). Negative control has all reagents except total RNA. MM is 100 bp DNA ladder. We found that BT-20, BT-549 and HCC1806 TNBC cell lines express LHRH-R mRNA while 3T3 normal fibroblasts have no expression. We have also found that patient-derived TNBC tumors BR1367 (PDX1), J000080739 (PDX2) and BR1126 (PDX3) express LHRH-R mRNA. Chem1-LHRH receptor over-expressing cells were used as positive control.

### Example 4

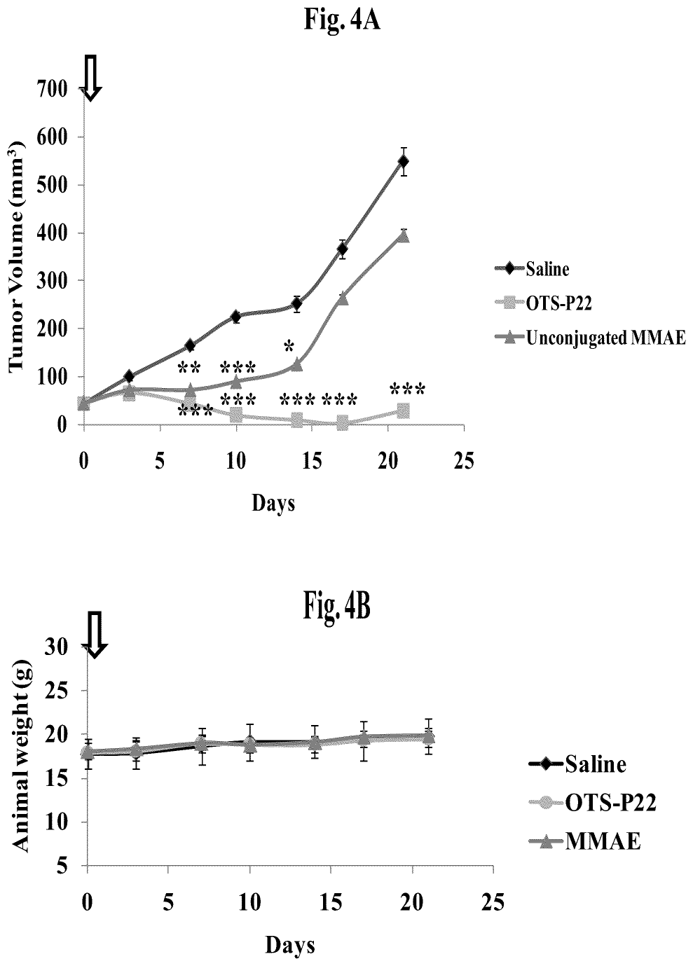

FIG. 4A depicts tumor volume in female SCID mice bearing HCC1806 TNBC CDX during treatment with saline; OTS-P22 or MMAE at a single i.v. injection of 0.5 μmol/kg on day 0 (arrow). Tumor volume values in mm³ are presented as mean±standard error of 10 mice per group. Statistical analysis was performed using ANOVA followed by Tukey's multiple comparison test. ***<0.001, **<0.01, *<0.05 were significant. We found that a single intravenous injection of OTS-P22 at 0.5 μmol/kg (1.435 mg/kg) given when the initial solid tumor volume was 44 mm³ caused a 100% tumor growth inhibition of HCC1806 TNBC CDX, 21 days post-treatment, with 7/10 mice showing complete response, 2/10 mice had partial responses of 80.5% and 1/10 mice had a partial response of 88.5% while MMAE had no significant effect.

FIG. 4B depicts animal weight in female SCID mice bearing HCC1806 TNBC CDX during treatment with saline; OTS-P22 or MMAE at a single i.v. injection of 0.5 μmol/kg on day 0 (arrow). Values in mm³ are presented as mean±standard error of 10 mice per group. Neither treatment with OTS-P22 or MMAE affected animal weight.

### Example 5

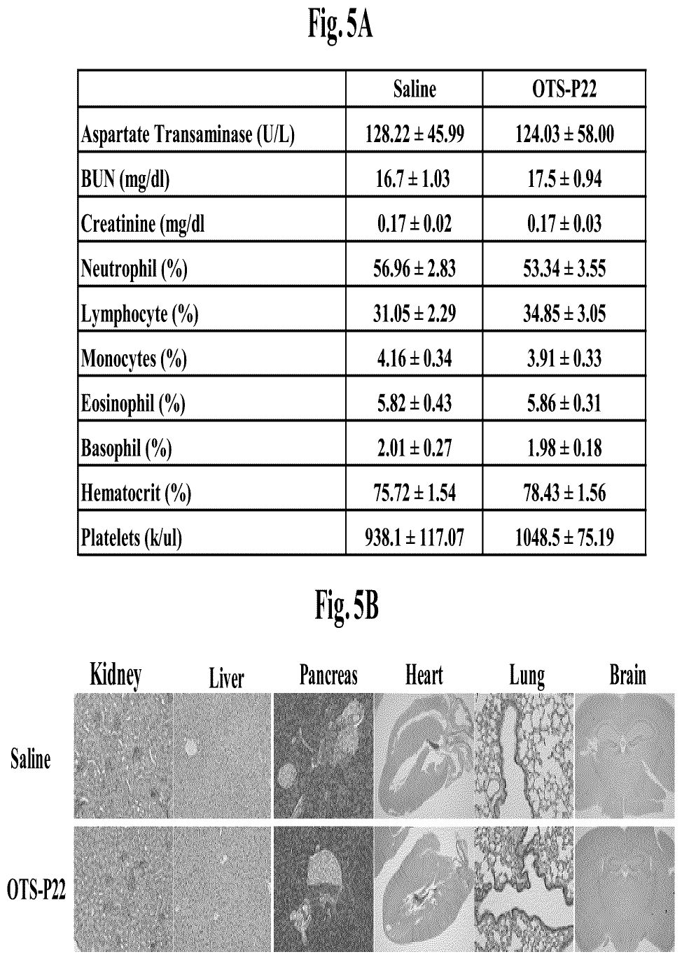

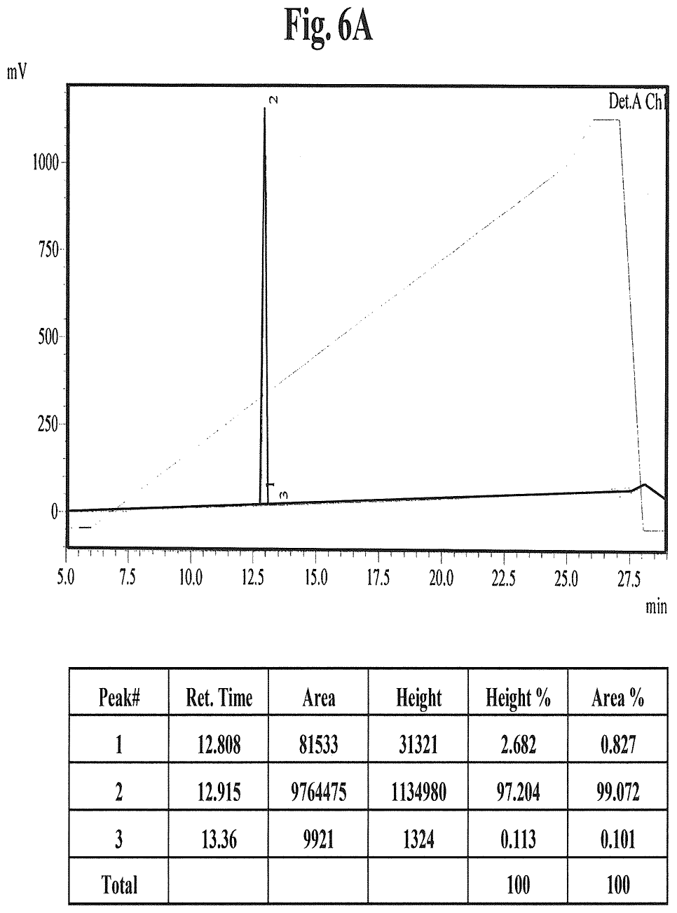

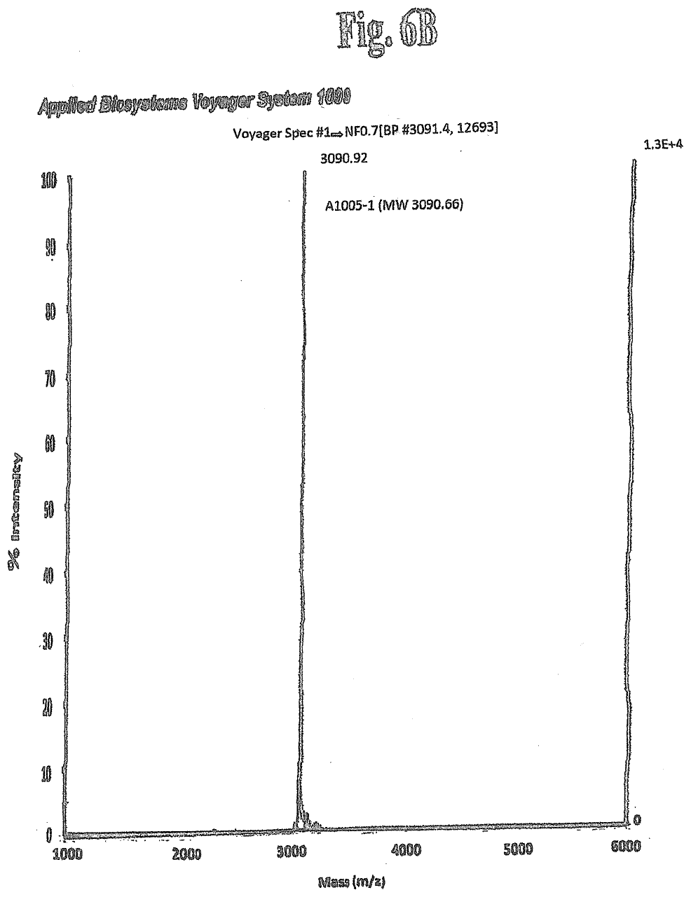

FIG. 5A depict hematology parameters of saline- and OTS-P22 treated SCID mice bearing HCC1806 TNBC xenografts (n=10) as described in FIG. 4A and into in accordance with the preferred embodiment of the present invention;

FIG. 5B depict representative hematoxylin-eosin staining slides of normal organs from saline- and OTS-P22 treated SCID mice bearing HCC1806 TNBC xenografts as described in FIG. 4A and into in accordance with the preferred embodiment of the present invention;

FIG. 6A depicts the RP-HPLC analysis of LHRH drug conjugate OTS-P23. The synthesis of the ligand Glp-His-DPal-Ser-Tyr-DLys(azide)-Leu-Arg-Pro-DAla-amide and was DBCO-dPEGg-Val-Cit-PABC-MMAE accomplished using Fmoc or Boc solid phase peptide synthesis (SPPS). The ligand was then conjugated through its DLysine (azide) to DBCO-dPEGg-Val-Cit-PABC-MMAE at 1/1.2 molar ratio in DMF at room temperature overnight by means of copper-free click chemistry and generating drug conjugate OTS-P23 with the structure Glp-His-DPal-Ser-Tyr-DLys[dPEG₈-Val-Cit-PABC-MMAE]-Leu-Arg-Pro-DAla-amide. The conjugation of DBCO to azide group generated a stable triazole bond. OTS-P23 was then purified according to the protocol described in FIG. 1. OTS-P23 was eluted at minute 12.915 with 99% purity.

FIG. 6B depicts the mass spectrometric analysis (MS) of drug conjugate OTS-P23 using Applied Biosystems Voyager System 1099 at 0.95 min run time and 1 ml/min flow rate. OTS-P23 has molecular weight of 3090.66 Dalton.

### Example 7

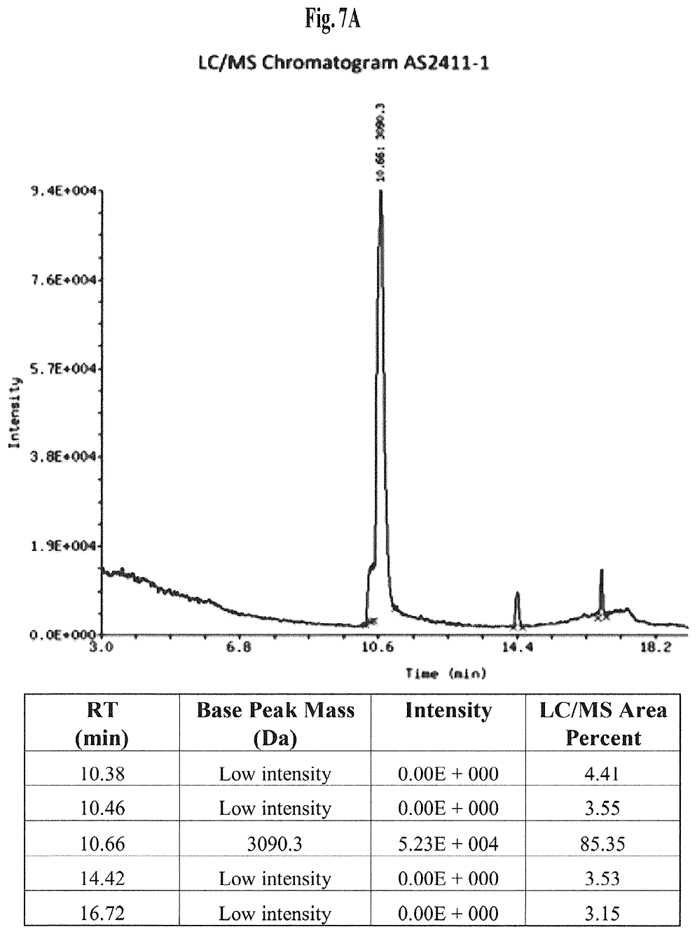

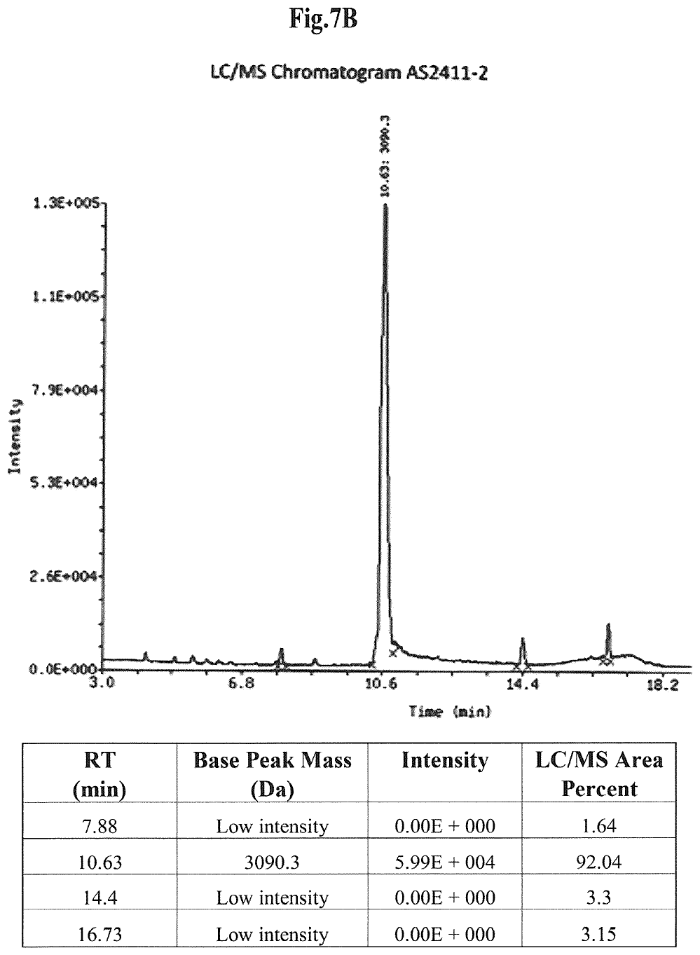

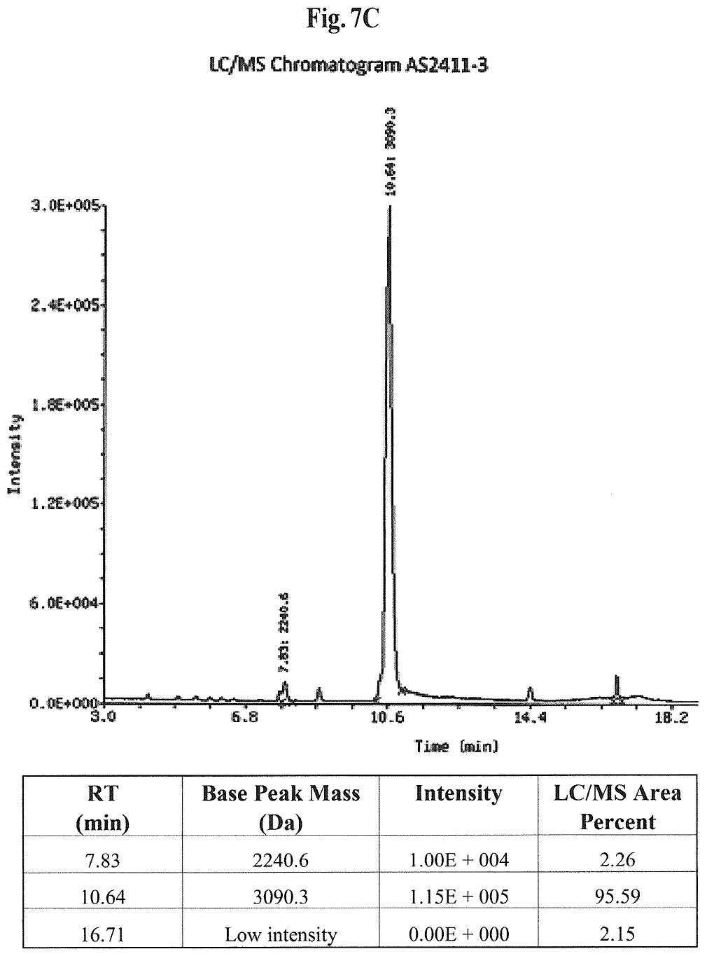

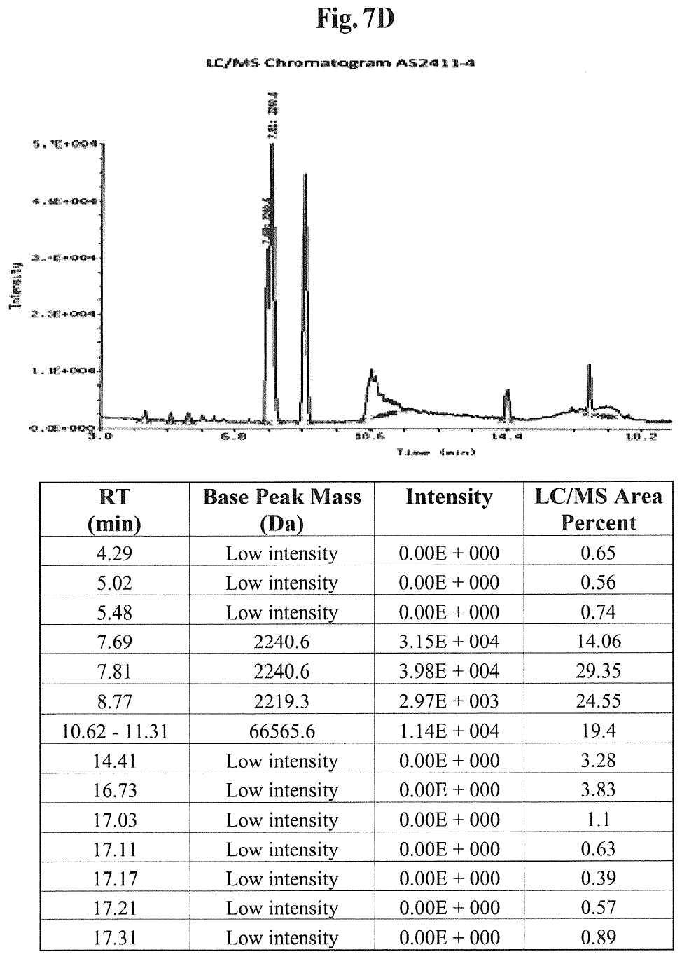

FIGS. 7A, 7B, 7C and 7D depicts the in-vitro stability of OTS-P23 in human plasma at 0, 2, 8 and 24 hours incubation times respectively in accordance with a preferred embodiment of the present invention. OTS-P23 is incubated at 100 μg/100 μl in 80% plasma from human or for 0, 2, 8 or 24 hours. Solutions are then centrifuged using Amicon centrifugal devices (cut-off 10 KDa) and 20 μl of the lower molecular weight fractions are analyzed by RP-HPLC/mass spectrometry. We found OTS-P22 to be highly stable in human plasma in-vitro with the majority of the conjugate (97.74%) still intact after 8 hours incubation. A minor peak (2.26%) was detected with a molecular weight of 2240.3 Dalton (metabolite).

Table 1 depicts competition binding IC₅₀ of [DTrp⁶]LHRH, OTS-P22 and OTS-P23 with ¹²⁵I[DTrp⁶]LHRH to membranes from Chem-1 cells over-expressing LHRH receptor in accordance with a preferred embodiment of the present invention.

### Example 8

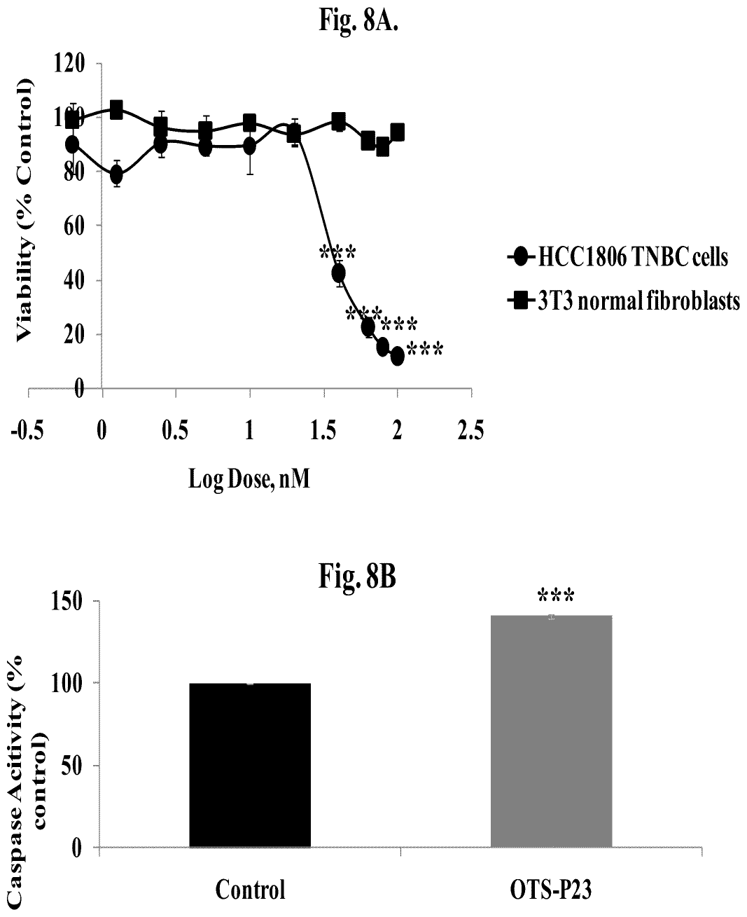

FIG. 8A depicts the effect of drug conjugate OTS-P23 on the viability of HCC1806 and normal 3T3 fibroblasts in an MTS proliferation assay in accordance with the preferred embodiment of the present invention.

FIG. 8B depicts the effect of OTS-P23 on caspase activity in HCC1806 cells in accordance with the preferred embodiment of the present invention.

### Example 9

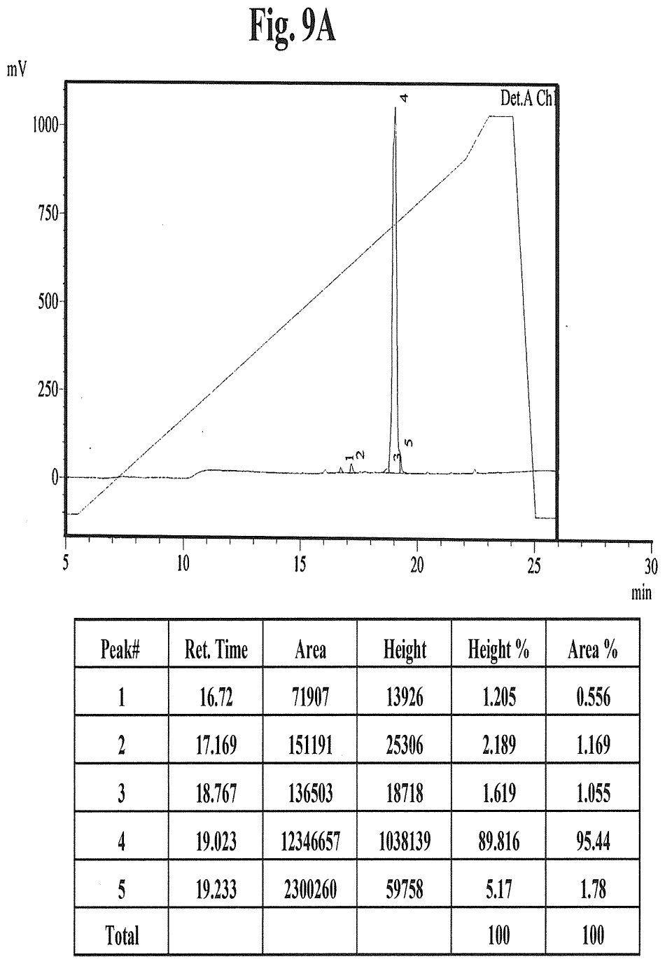

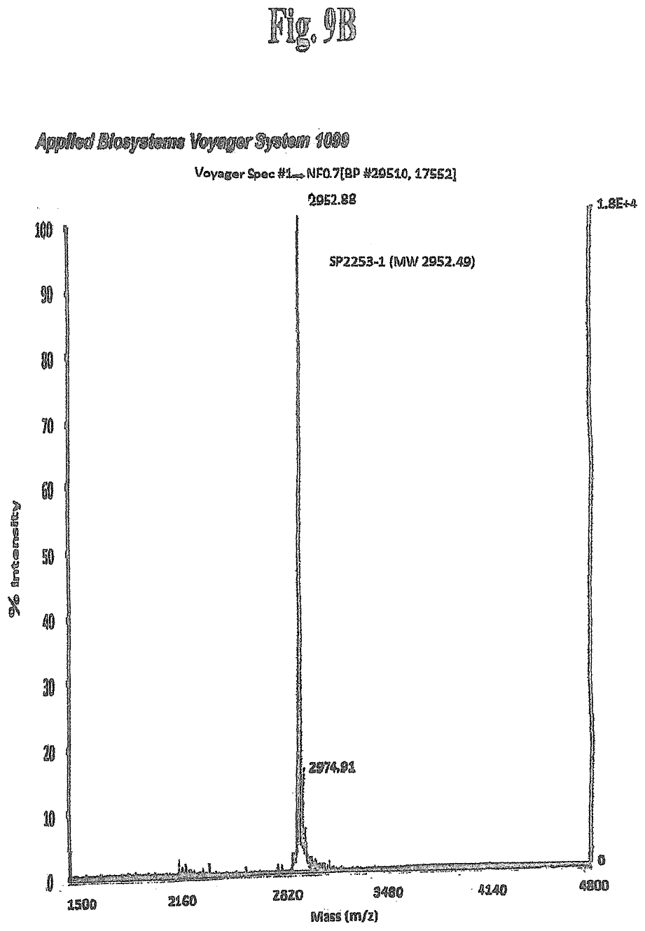

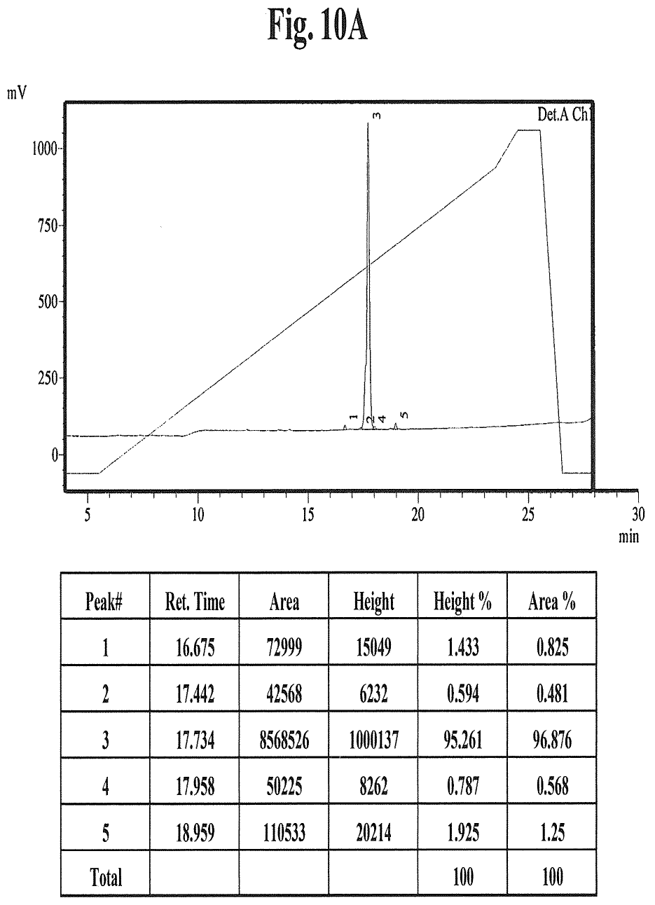

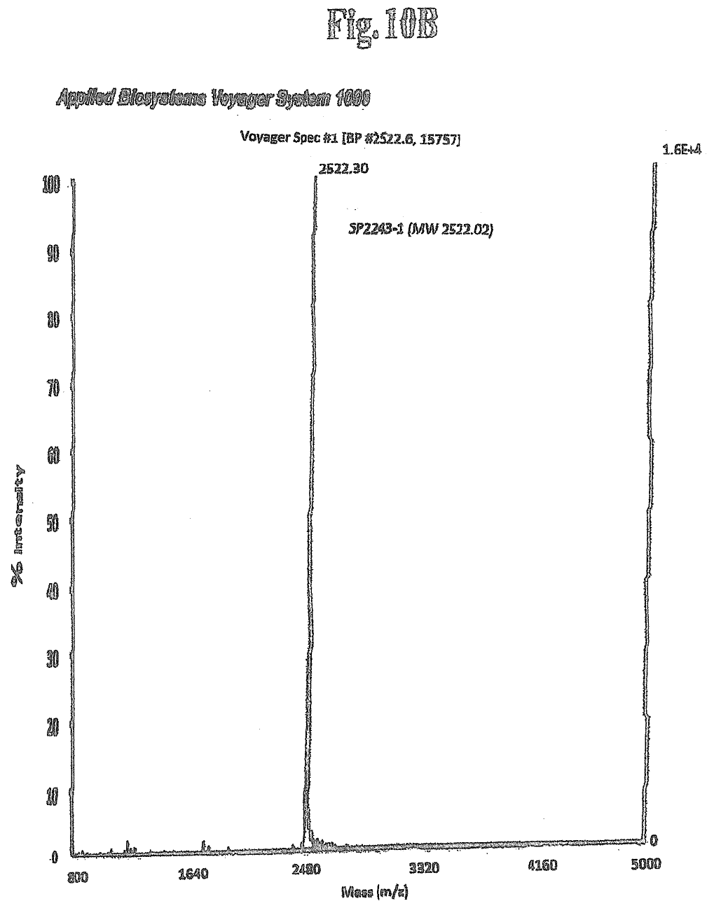

FIG. 9A depicts the RP-HPLC analysis of LHRH drug conjugate OTS-P23b. The synthesis of the ligand Glp-His-Trp-DSer-Tyr-DLys(azide)-Leu-Arg-Pro-Ethylamide and DBCO-dPEG₅-Val-Cit-PABC-MMAE was accomplished using Fmoc or Boc solid phase peptide synthesis (SPPS). The ligand was then conjugated through its DLysine (azide) to DBCO-dPEG₅-Val-Cit-PABC-MMAE at 1/1.2 molar ratio in DMF at room temperature overnight by means of copper-free click chemistry and generating drug conjugate OTS-P23b with the structure Glp-His-Trp-DSer-Tyr-DLys[dPEG₅-Val-Cit-PABC-MMAE]-Leu-Arg-Pro-Ethylamide. The conjugation of DBCO to azide group generated a stable triazole bond. OTS-P23b was then purified according to the protocol described in FIG. 1. OTS-P23b was then purified according to the protocol described in FIG. 1. OTS-P23b was eluted at minute 19.023 min with a purity of 95%.

FIG. 9B depicts the mass spectrometric analysis (MS) of drug conjugate OTS-P23b using Applied Biosystems Voyager System 1099 at 0.95 min run time and 1 ml/min flow rate. OTS-P23b has molecular weight of 2952.49 Dalton.

### Example 10

FIG. 10A depicts the RP-HPLC analysis of LHRH drug conjugate OTS-P26. The synthesis of the ligand Glp-His-DPal-Ser-Tyr-DCys-Leu-Arg-Pro-DAla-amide and MC-Val-Cit-PABC-MMAE was accomplished using Fmoc or Boc solid phase peptide synthesis (SPPS). The ligand was then conjugated through its thiol group of DCysteine to the maleimidocaproil end of MC-Val-Cit-PABC-MMAE at 1/1.2 molar ratio in DMF at room temperature overnight and generating drug conjugate OTS-P26 with the structure Glp-His-DPal-Ser-Tyr-DCys[Val-Cit-PABC-MMAE]-Leu-Arg-Pro-DAla-amide. The conjugation of thiol to MC group generated a stable thioether bond. OTS-P26 was then purified according to the protocol described in FIG. 1. OTS-P26 was then purified according to the protocol described in FIG. 1. OTS-P26 was eluted at minute 17.734 with a purity of 96.9%.

FIG. 10B depicts the mass spectrometric analysis (MS) of drug conjugate OTS-P26 using Applied Biosystems Voyager System 1099 at 0.95 min run time and 1 ml/min flow rate. OTS-P26 has molecular weight of 2522.02 Dalton.

### Example 11

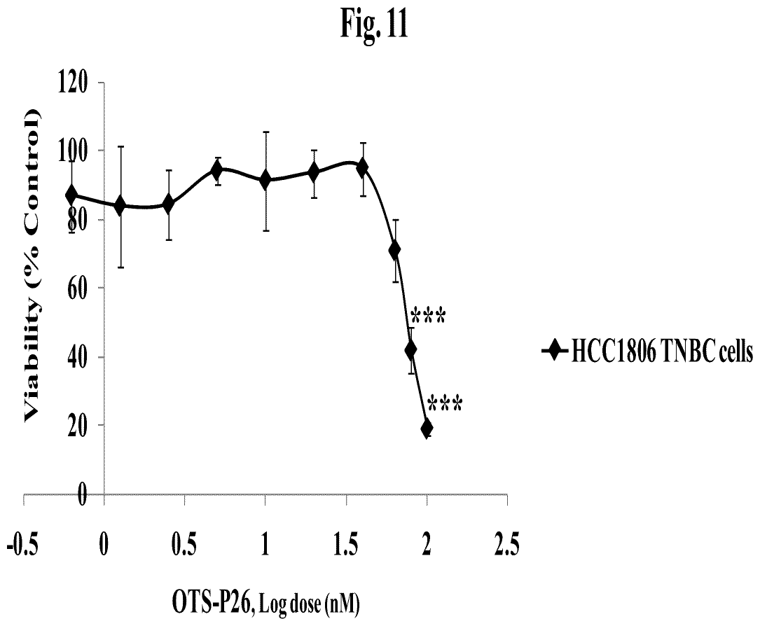

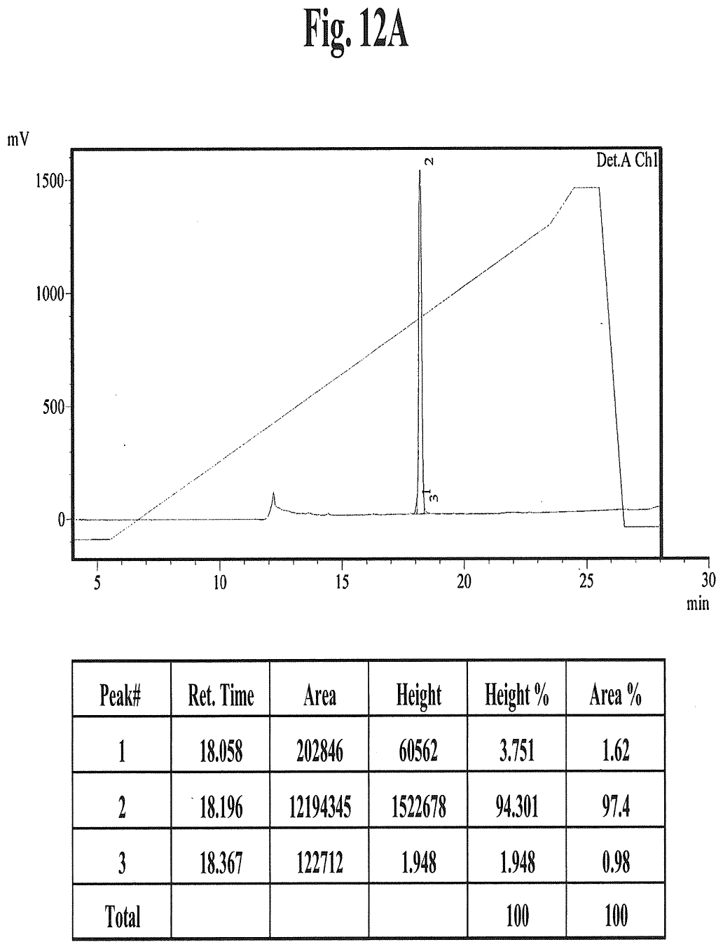

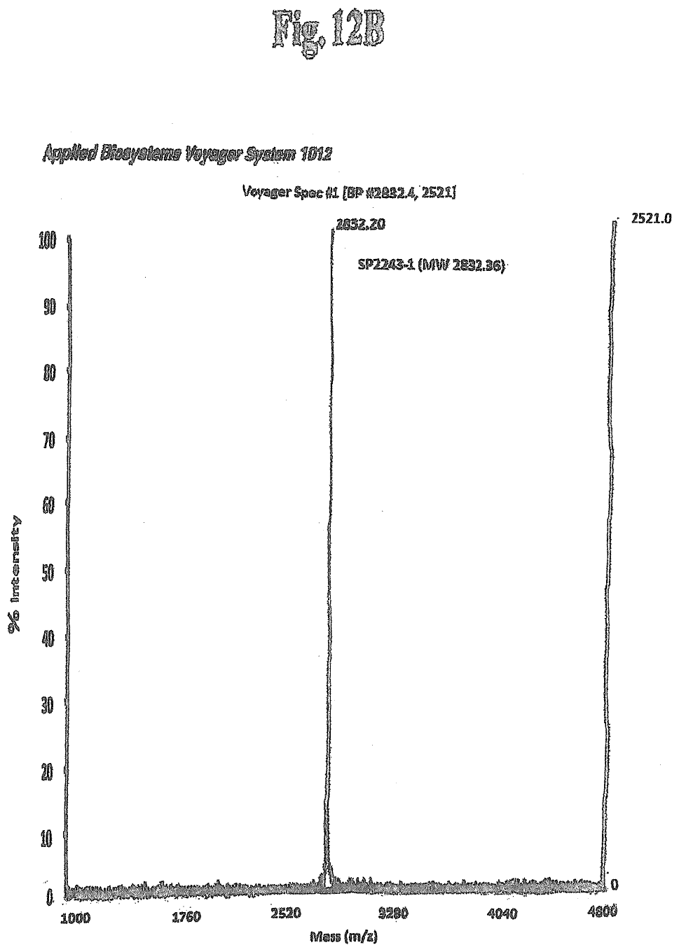

FIG. 11 depicts the effect of drug conjugate OTS-P26 on the viability of HCC1806 TNBC cells in an MTS proliferation assay in accordance with the preferred embodiment of the present invention. OTS-P26 reduces the viability of HCC1806 TNBC cells in a dose-dependent manner.

### Example 12

FIG. 12A depicts the RP-HPLC analysis of LHRH drug conjugate OTS-P28. The synthesis of the ligand Glp-His-DPal-Ser-Tyr-DCys-Leu-Arg-Pro-DAla-amide and Mal-dPEGg-Val-Cit-PABC-MMAE was accomplished using Fmoc or Boc solid phase peptide synthesis (SPPS). The ligand was then conjugated through its thiol group of DSerine to the maleimide end of Mal-dPEGg-Val-Cit-PABC-MMAE at 1/1.2 molar ratio in DMF at room temperature overnight by means of copper-free click chemistry and generating drug conjugate OTS-P26 with the structure Glp-His-DPal-Ser-Tyr-DCys[dPEG₈-Val-Cit-PABC-MMAE]-Leu-Arg-Pro-DAla-amide. The conjugation of thiol to Maleimide group generated a stable thioether bond. OTS-P28 was then purified according to the protocol described in FIG. 1. OTS-P28 was eluted at minute 18.196 with a purity of 97.4%.

FIG. 12B depicts the mass spectrometric analysis (MS) of drug conjugate OTS-P28 using Applied Biosystems Voyager System 1099 at 0.95 min run time and 1 ml/min flow rate. OTS-P28 has molecular weight of 2832.36 Dalton.

### Example 13

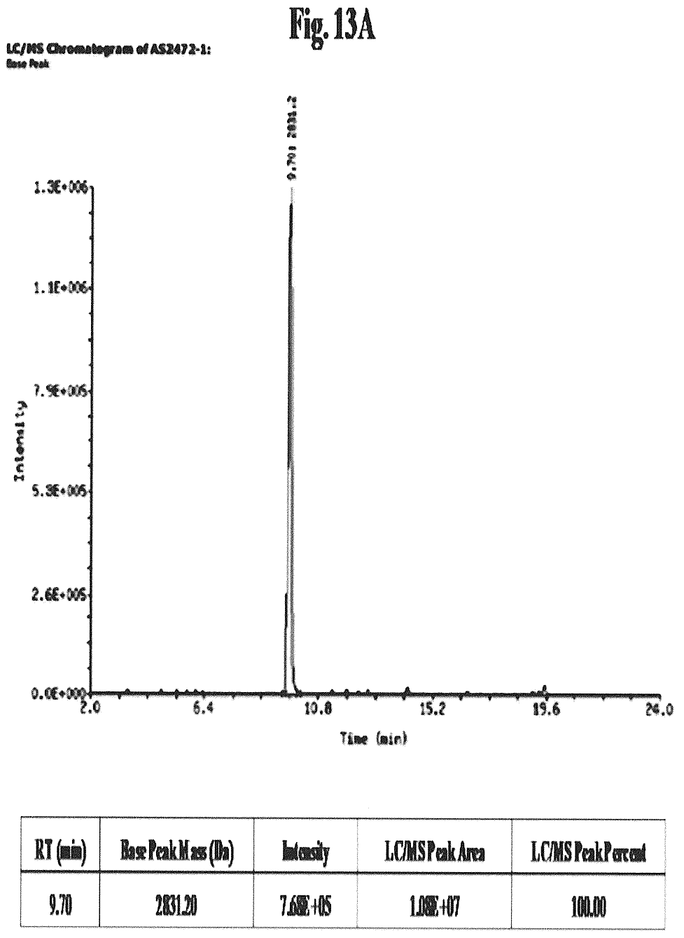

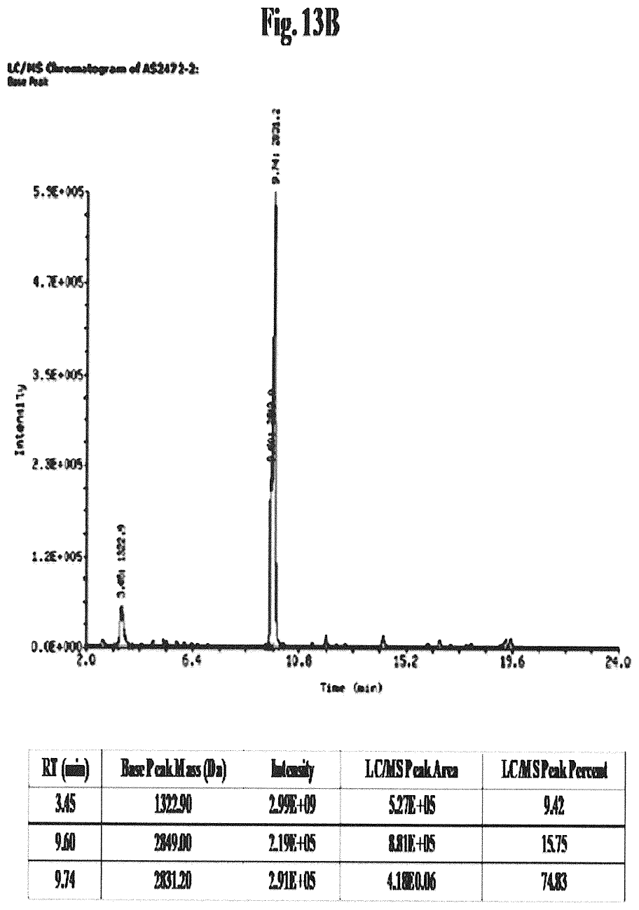

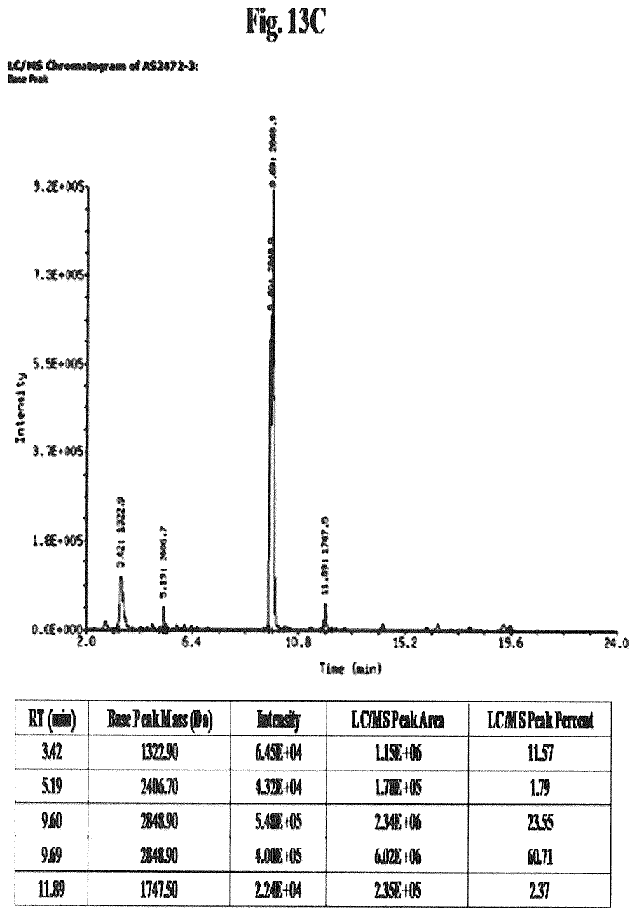

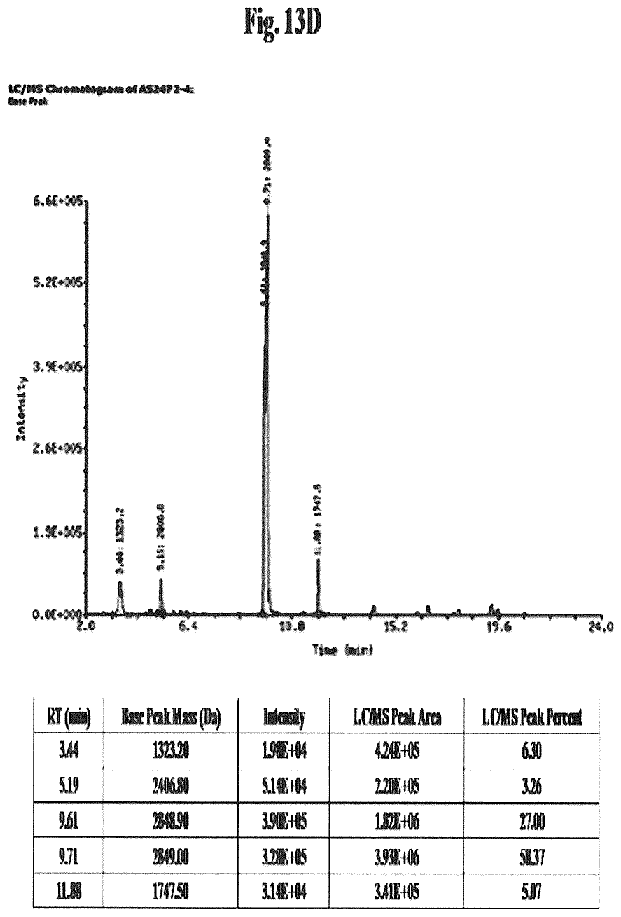

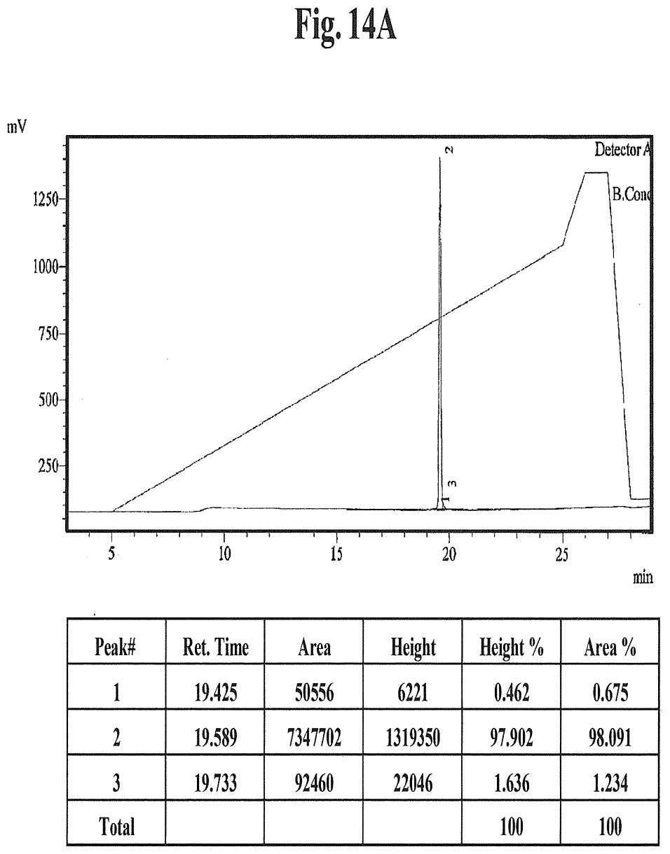

FIGS. 13A, 13B, 13C and 13D depicts the in-vitro stability of OTS-P28 in human plasma at 0, 2, 8 and 24 hours incubation times respectively in accordance with a preferred embodiment of the present invention. OTS-P23 is incubated at 100 μg/100 μl in 80% plasma from human or for 0, 2, 8 or 24 hours. Solutions are then centrifuged using Amicon centrifugal devices (cut-off 10 KDa) and 20 μl of the lower molecular weight fractions are analyzed by RP-HPLC/mass spectrometry. We found OTS-P22 to be highly stable in human plasma in-vitro with the majority of the conjugate (97.74%) still intact after 8 hours incubation. A minor peak (2.26%) was detected with a molecular weight of 2240.3 Dalton (metabolite).

### Example 14

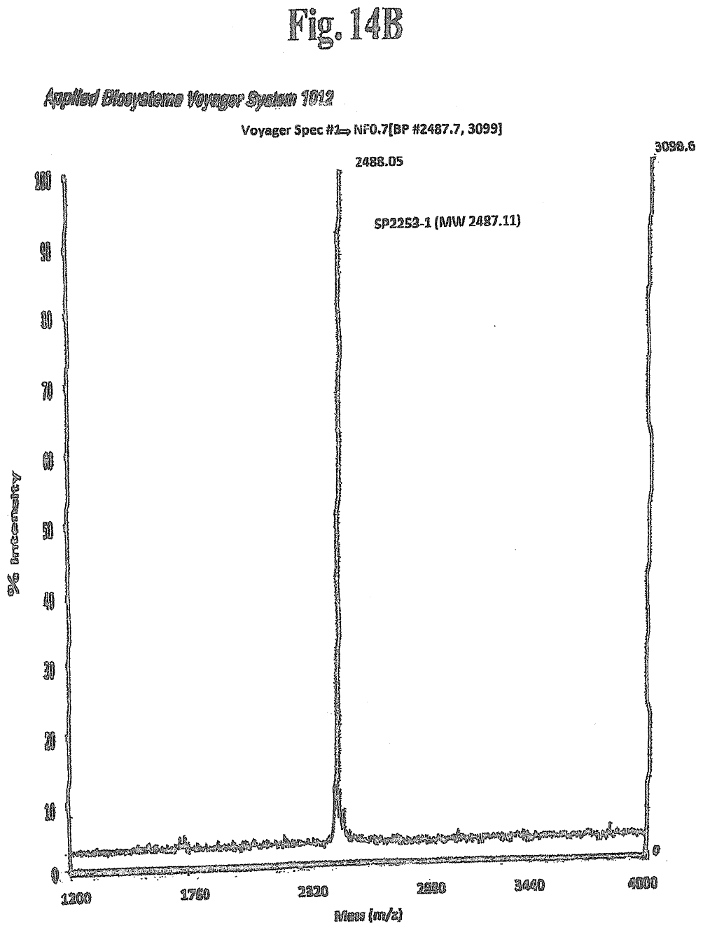

FIG. 14A depicts the RP-HPLC analysis of LHRH drug conjugate OTS-P31. The synthesis of the ligand Glp-DHis-Trp-DSer-Tyr-DLys-Leu-Arg-Pro-DAla-amide and OSu-Glut-Val-Cit-PABC-MMAE was accomplished using Fmoc or Boc solid phase peptide synthesis (SPPS). The ligand was then conjugated through its NH₂ side chain group of DLysine to the OSu end of OSu-Glut-Val-Cit-PABC-MMAE at 1/1.2 molar ratio in DMF at room temperature overnight and generating drug conjugate OTS-P31 with the structure Glp-DHis-Trp-DSer-Tyr-DLys[Glut-Val-Cit-PABC-MMAE]-Leu-Arg-Pro-DAla-amide. The conjugation of OSu to NH₂ side chain generated a stable amide bond. OTS-P31 was then purified according to the protocol described in FIG. 1. OTS-P31 was eluted at minute 19.589 with a purity of 98.09%.

FIG. 14B depicts the mass spectrometric analysis (MS) of drug conjugate OTS-P31 using Applied Biosystems Voyager System 1099 at 0.95 min run time and 1 ml/min flow rate. OTS-P31 has molecular weight of 2488.06 Dalton.

### Example 15

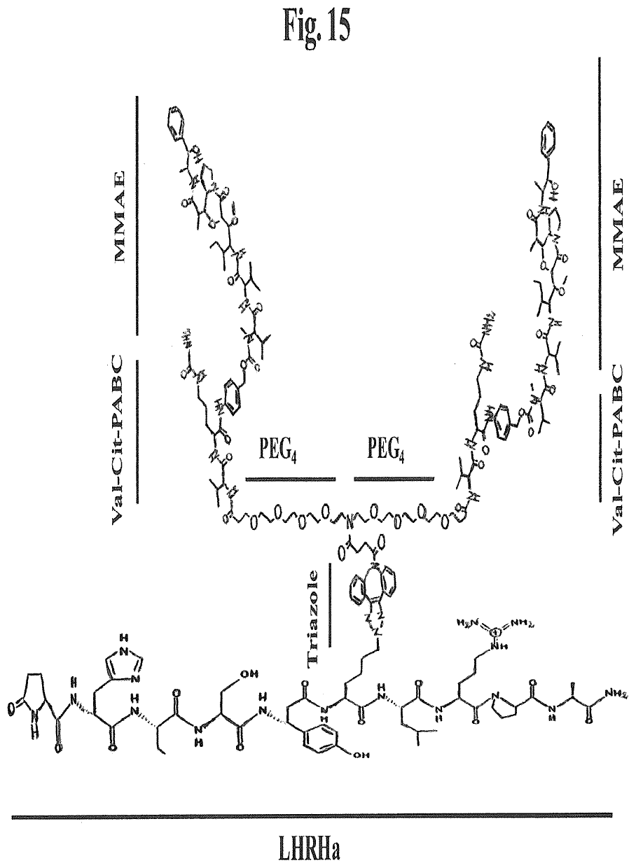

FIG. 15 depicts schematic of LHRHa carrying two copies of Val-Cit-PABC-MMAE via DBCO-N-bis(PEG₄-NHS ester) branched spacer. First, DBCO-N-bis(PEG₄-NHS ester) branched spacer is conjugated to NH₂—Val-Cit-PABC-MMAE at 1/3 molar ratio in phosphate buffer (pH 7.2) for 4 hours at room temperature. The resulting product is purified using RP-HPLC, then subject to second conjugation to LHRHa at N₃ side chain of DLysine[^{N3] 6} at a 1.2/1 molar ratio in DMF at room temperature overnight by means of copper-free click chemistry. The final peptide conjugate is purified using RP-HPLC and mass spectrometry.

### Example 16

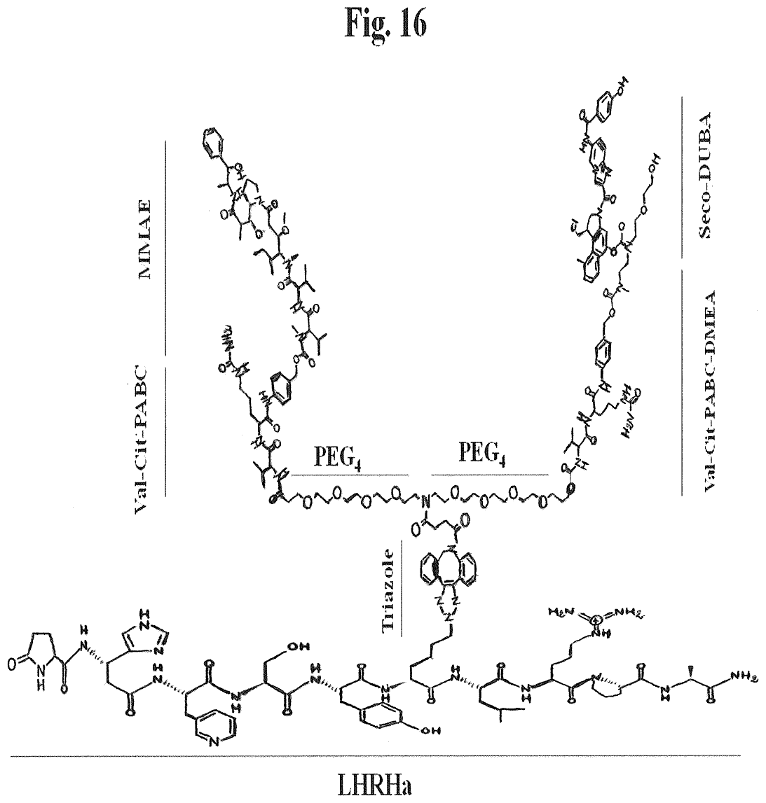

FIG. 16 depicts schematic of LHRHa carrying one copy of MMAE (tubulin polymerization inhibitor) via Val-Cit-PABC linker and one copy of seco-DUBA (DNA alkylating agent) via Val-Cit-PABC-DMEA linker using DBCO-N-bis(PEG₄-NHS ester) branched spacer. First, DBCO-N-bis(PEG₄-NHS ester) branched spacer is conjugated to NH₂—Val-Cit-PABC-MMAE at 5/1 molar ratio in phosphate buffer (pH 7.2) for 4 hours at room temperature. The resulting product is analyzed using RP-HPLC/MS, then subject to second conjugation to NH₂—Val-Cit-PABC-DMEA-seco-DUBA in phosphate buffer (pH 7.2) for 4 hours at room temperature. The resulting product is analyzed using RP-HPLC/MS, then subject to a third conjugation to LHRHa at N₃ side chain of DLysine [^{N3}]6 at a 1.2/1 molar ratio in DMF at room temperature overnight by means of copper-free click chemistry. The final peptide conjugate is analyzed using RP-HPLC/MS.

### Example 17

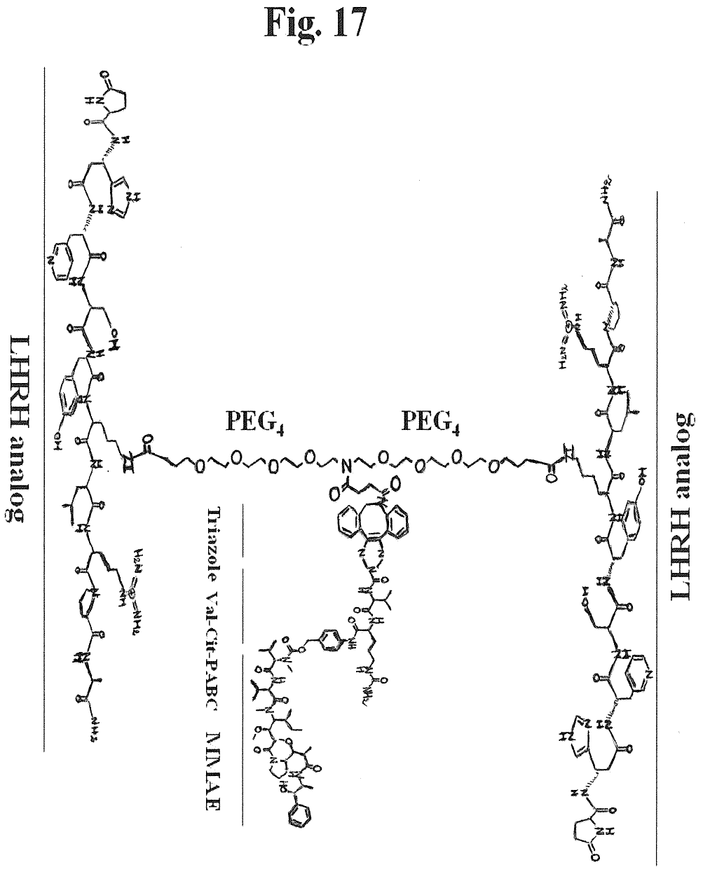

FIG. 17 depicts schematic of dimeric LHRHa carrying two copies of LHRHa and one copy of Val-Cit-PABC-MMAE via DBCO-N-bis(PEG₄-NHS ester) branched spacer. First, DBCO-N-bis(PEG₄-NHS ester) branched spacer is conjugated via its NHS ester ends to NH₂ side chain of DLysine⁶ LHRHa at a 1/3 molar ratio in phosphate buffer (pH 7.2) for 4 hours at room temperature generating stable amide bonds. The resulting product is analyzed using RP-HPLC/MS, then conjugated to N₃-PEG₃-Val-Cit-PABC-MMAE at 1/1.2 molar ratio in DMF at room temperature overnight using copper-free click chemistry and generating a stable triazole bond.

This application incorporates by reference the revised sequence listing submitted on 2 compact discs and on paper as an ASCII text file (718 bytes) on 9/25/2023, titled “Sequence Listing”.

### Claims

What is claimed are specific conjugation chemistries to LHRH analogs to generate monomeric or multimeric peptide drug conjugates (PDCs) carrying a single or multiple payloads. Conjugations through amino acids in position 4, 6 or 8 of LHRH analogs (LHRHa) generate highly stable and potent PDCs. Amino acid substitutions are introduced to LHRHa in order to increase their stability in the bloodstream. In addition, four conjugation chemistries are used to generate the PDCs with highly stable triazole, thioether, oxime or amide bonds. The spacers used are for increasing solubility in aqueous solutions and for providing sufficient distance for the linker to be recognized and cleaved by cathepsins in the lysosomes. The presence of peptidic linkers which are stable in the bloodstream but cleaved inside the lysosomes of cancer cells by cathepsin B allows the release of attached payloads leading to their exit from the lysosomal department into the cytoplasm to exert their anti-proliferative and pro-apoptotic activities. Thus, generated PDCs are expected to be more stable, potent and safer than previous PDCs or small molecule drug conjugates targeting the same receptors. Below are examples of methods of making and using of such peptide drug conjugates:

LHRH Ligands:

1. A peptide ligand having the following structure containing 9 or 10 amino acids:
Xaa₁-Xaa₂-Xaa₃-Xaa₄-Xaa₅-Xaa₆-Xaa₇-Xaa₈-Xaa₉-Xaa₁₀-amide
Xaa₁-Xaa₂-Xaa₃-Xaa₄-Xaa₅-Xaa₆-Xaa₇-Xaa₈-Xaa₉-NHEt
Xaa₁ is Glp (pyroglutamic acid) or Ac-D₂-Nal (N-acetyl-3-naphthalen-2-yl),
Xaa₂ is His (Histidine), D-His (D-Histidine) or D₄-Cpa (4-chloro-D-Phenylalanine),
Xaa₃ is Trp (Tryptophan), D-Trp or D-Pal (3-pyridyl-D-Alanine),
Xaa₄ is Ser (Serine), D-Ser (D-Serine), D-Lys (D-Lysine), D-Lys^{[N3]} (D-Lysine with N₃ side chain), Lys-Aoa (Lysine-aminooxyacetyl), D-Cys (D-Cysteine),
Xaa₅ is Tyr (Tyrosine) or NMe-Tyr (N-methyl-Tyrosine),
Xaa₆ is D-Lys (D-Lysine), D-Lys^{[N3]} (D-Lysine with N₃ side chain), Lys-Aoa (Lysine-aminooxyacetyl), D-Cys (D-Cysteine), D-Glu (D-Glutamic acid) or D-Asp (D-Aspartic acid),
Xaa₇ is Leu (Leucine), D-Leu (D-Leucine), NLe (Norleucine), Trp (Tryptophan) or D-Trp (D-Tryptophan),
Xaa₅ is Arg (Arginine), Cit (Citrulline), Tyr (Tyrosine), D-Tyr (D-Tyrosine), Lys (Lysine), D-Lys, D-Lys [N₃] (Lysine with azide side chain), Lys-Aoa (Lysine-aminooxyacetyl), Cys (Cysteine) or D-Cys.
Xaa₉ is Pro (Proline) or D-Pro (D-Proline),
Xaa₁₀ is Gly (Glycine), D-Ala (D-Alanine), D-AzaGly (D-AzaGlycine) or Des-Gly (deleted Glycine),
NHEt is NH-Ethylamide.

2. A compound comprising: Monomeric or multimeric LHRH analogs (I, II, III) with the structure according to claim 1; a single or multiple cytotoxic agents; a spacer comprising linear or branched discrete polyethylene glycol (dPEG), glutaric acid or capric acid; a linker comprising valine-Citrulline-PABC, Val-Ala, Gly-Gly-Phe-Gly or other peptide linkers; a highly stable bond comprising a triazole wherein the bond is formed after conjugation of DBCO-spacer-linker-payload to an azide group contained at D-lysine(N₃) in position 4, 6 or 8 of the LHRH ligand. PABC is p-aminobenzyloxycarbonyl. DBCO is dibenzylcyclooctyne.

3. A compound comprising: Monomeric or multimeric LHRH analogs (I, II, III) with the structure according to claim 1; a single or multiple cytotoxic agents; a spacer comprising linear or branched discrete polyethylene glycol (dPEG); valine-Citrulline-PABC (aminobenzyloxycarbonyl), Val-Ala, Gly-Gly-Phe-Gly or other cleavable linkers; a highly stable bond comprising a thioether bond wherein the bond is formed after conjugation of spacer-linker-payload to the thiol group of D-Cysteine in position 4, 6 or 8 of the ligand. Spacer is maleimidocaproyl (MC) or Mal-dPEGₙ (n=1-36).

4. A compound comprising: Monomeric or multimeric LHRH analogs (I, II, III) with the structure according to claim 1; a single or multiple cytotoxic agents; a spacer comprising linear or branched discrete polyethylene glycol (dPEG); valine-Citrulline-PABC, Val-Ala, Gly-Gly-Phe-Gly or other cleavable linkers; a highly stable bond comprising an amide bond wherein the bond is formed after conjugation of NHS ester-spacer-linker-toxin to the NH₂ side chain group of D-Lysine in position 4, 6 or 8 of the ligand.

5. A compound comprising: Monomeric or multimeric LHRH analogs (I, II, III) with the structure according to claim 1; a single or multiple cytotoxic agents; a spacer comprising linear or branched discrete polyethylene glycol (dPEG); valine-Citrulline-PABC, Val-Ala, Gly-Gly-Phe-Gly or other cleavable linkers; a highly stable bond comprising an oxime bond wherein the bond is formed after conjugation of Aldehyde-spacer-linker-toxin or Ketone-spacer-linker-toxin to the aminoxyacetyl side chain group of D-Lysine-Aoa in position 4, 6 or 8 of the ligand.

6. A compound comprising bispecific drug conjugates containing a copy of LHRH analogs (I, II, III) with the structure according to claim 1 and a copy of bombesin analog (bispecific LHRHa-bombesin analog), or somatostatin analog (bispecific LHRHa-somatostatin analog); a cytotoxic agent; a branched discrete polyethylene glycol (dPEG) including but not limited to DBCO-N-bis(PEG₄-NHS ester); linkers including valine-Citrulline-PABC, Val-Ala, Gly-Gly-Phe-Gly; a highly stable bond comprising an amide, triazole, thioether or oxime bond wherein the bond is formed after conjugation of NH₂, N₃ or aminoxyacetyl side chains of D-Lysine in position 4, 6 or 8 of the ligand with NHS ester, DBCO, or ketone/aldehyde reactive groups respectively.

7. Example of payloads in the compound could be MMAE, MMAF, MMAD, tubulysin, DM1 (Mertansine), PNU-159682 (doxorubicin metabolite), Duocarmycins, seco-DUBA, PBD (Pyrrolobenzodiazepine), SG3199 (Pyrrolobenzodiazepine dimer), DX8951 (Exatecan) or other highly potent antitumor payloads.

8. A method for slowing the growth and lessening the incidents of metastasis of cancer cells in a mammal; wherein the cancer cells express a receptor for luteinizing hormone-releasing hormone;
the method comprising administering to the recipient an effective amount and an effective route of the compound of claim 1.

9. A method for slowing the growth and lessening the incidents of metastasis of cancer cells in a mammal; wherein the cancer cells express a receptor for luteinizing hormone-releasing hormone; the method comprising administering to the recipient an effective amount and an effective route of the compound of claim 2.

10. A method for slowing the growth and lessening the incidents of metastasis of cancer cells in a mammal; wherein the cancer cells express a receptor for luteinizing hormone-releasing hormone; the method comprising administering to the recipient an effective amount and an effective route of the compound of claim 3.

11. A method for slowing the growth and lessening the incidents of metastasis of cancer cells in a mammal; wherein the cancer cells express a receptor for luteinizing hormone-releasing hormone; the method comprising administering to the recipient an effective amount and an effective route of the compound of claim 4.

12. A method for slowing the growth and lessening the incidents of metastasis of cancer cells in a mammal; wherein the cancer cells express a receptor for luteinizing hormone-releasing hormone; the method comprising administering to the recipient an effective amount and an effective route of the compound of claim 5.

13. A method for slowing the growth and lessening the incidents of metastasis of cancer cells in a mammal; wherein the cancer cells express a receptor for luteinizing hormone-releasing hormone; the method comprising administering to the recipient an effective amount and an effective route of the compound of claim 6.

14. A method for slowing the growth and lessening the incidents of metastasis of cancer cells in a mammal; wherein the cancer cells express a receptor for luteinizing hormone-releasing hormone; the method comprising administering to the recipient an effective amount and an effective route of compounds containing payloads from claim 7.
